(19) United States
(12) Patent Application Publication
(10) Pub. No.: US 2021/0347884 A1
(43) Pub. Date: Nov. 11, 2021

(54) METHODS AND COMPOSITIONS FOR TREATING A BRAIN INJURY

(71) Applicant: THE GENERAL HOSPITAL CORPORATION, Boston, MA (US)

(72) Inventor: William Taylor KIMBERLY, Belmont, MA (US)

(73) Assignee: THE GENERAL HOSPITAL CORPORATION, Boston, MA (US)

(21) Appl. No.: 17/197,380

(22) Filed: Mar. 10, 2021

Related U.S. Application Data
(62) Division of application No. 16/486,687, filed on Aug. 16, 2019, now abandoned.
(60) Provisional application No. 62/460,229, filed on Feb. 17, 2017.

(51) Int. Cl. C07K 16/28; G01N 33/68

(52) U.S. Cl. CPC C07K 16/28; G01N 2800/52; G01N 2800/2871; G01N 33/6896

(57) ABSTRACT

Disclosed herein are methods and compositions for the treatment of a brain injury. The method involves administering to a subject who has had a brain injury an agent that inhibits sST2, or an agent that upmodulates IL-33. Methods for preventing a poor functional prognosis, as well as identifying a subject at risk of a poor functional prognosis following a brain injury are also provided herein.

### CROSS-REFERENCE TO RELATED APPLICATIONS

This application is a Divisional Application of U.S. Ser. No. 16/486,687 filed Aug. 16, 2019, which is a 35 U.S.C. § 371 National Phase Entry Application of International Application No. PCT/US2018/018537 filed Feb. 17, 2018, which designates the U.S. and claims benefit under 35 U.S.C. § 119(e) of U.S. Provisional Application No. 62/460,229 filed Feb. 17, 2017, the contents of which are incorporated herein by reference in their entirety.

### GOVERNMENT SUPPORT

This invention was made with Government support under Grant No. K23NS076597 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Feb. 26, 2021, is named 030258-088941-PCT_SL.txt and is 4,106 bytes in size.

### FIELD OF THE INVENTION

The field of the invention relates to methods and compositions for the treatment of a brain injury.

### BACKGROUND

Stroke is a leading cause of long-term disability, yet accurately predicting functional outcome after stroke remains challenging^{1,2}. Although clinical factors such as age, sex and stroke severity can stratify risk, a reliable blood-based biomarker at the time of stroke onset would help identify high-risk individuals and potentially inform treatment decisions.³ Furthermore, a stroke biomarker that could identify patients at greater risk for secondary ischemic injury, poor functional outcome and mortality would aid in prognosis.

### SUMMARY

The invention described herein is based in part on the discovery that soluble suppression of tumorigenicity 2 (sST2) serves as a prognostic biomarker for the functional outcome of a patient having suffered an ischemic stroke. Presented herein are data that indicate that increased levels of sST2 in a plasma sample taken from a subject 1 day and 5 days following a brain injury (e.g., an ischemic stroke) was independently associated with poor outcome and mortality within 90 days of the initial brain injury. Moreover, increased levels of sST2 in a plasma sample taken after a brain injury was an indicator for the likelihood of hemorrhagic transformation following ischemic stroke. Further, presented herein are data showing that sST2 serves as a prognostic biomarker for a poor functional outcome following a subarachnoid hemorrhage and intracerebral hemorrhage.

Increased levels of sST2 results in a pro-inflammatory response that is responsible for increased and sustained damage following a brain injury (e.g., ischemic stroke). It is specifically contemplated herein to administer to a subject who has had a brain injury an agent that inhibits sST2, or an agent that upmodulates IL-33, to prevent damage caused by a brain injury, reverse damage caused by a brain injury, and/or prevent a poor functional outcome following a brain injury.

Accordingly, one aspect of the invention described herein provides a method to improve clinical outcome after a brain injury in a subject comprising administering to the subject an agent that inhibits sST2.

Another aspect of the invention described herein provides a method to improve clinical outcome after a brain injury in a subject comprising administering to the subject an agent that upmodulates interleukin-33 (IL-33).

In one embodiment of any aspect, the brain injury is a cerebral stroke, a subarachnoid hemorrhage, a focal brain injury, or a traumatic brain injury. Exemplary cerebral strokes include, but are not limited to, acute ischemic stroke, transient ischemic attack, and hemorrhagic stroke. Exemplary focal brain injuries include, but are not limited to, an intraventricular hemorrhage, a subdural hemorrhage, an intracerebral hemorrhage, a cerebral contusion, a cerebral laceration, and an epidural hemorrhage. Exemplary traumatic brain injuries include, but are not limited to, coup-contrecoup brain injury, concussion, diffuse axonal injury, brain contusions, second impact syndrome, shaken baby syndrome, and penetrating injury.

In one embodiment of any aspect, the agent that inhibits sST2 is a small molecule, an antibody, a peptide, an antisense oligonucleotide, a genome editing system, and an RNAi. Exemplary RNAi include, but are not limited to, a microRNA, an siRNA, or a shRNA. In one embodiment of any aspect, the agent is an anti-sST2 antibody for therapeutic use.

In one embodiment of any aspect, the agent that upmodulates IL-33 is a small molecule, a peptide, or an expression vector encoding IL-33.

In one embodiment of any aspect, the agent is comprised in a vector. In one embodiment of any aspect, the vector is non-integrative or integrative.

In one embodiment of any aspect, any of the agents described herein are administered within 48 hours of the brain injury occurring.

In one embodiment of any aspect, any of the agents described herein are administered within 72 hours, within 96 hours, within 120 hours, within 144 hours, within 168 hours of the brain injury occurring.

In one embodiment of any aspect, inhibiting sST2 is decreasing the level and/or activity of sST2. In one embodiment of any aspect, the level and/or activity of sST2 is decreased by at least 50%, at least 60%, at least 70%, at least 80%, at least 90%, at least 99%, or more as compared to an appropriate control.

In one embodiment of any aspect, upmodulating IL-33 is increasing the level and/or activity of IL-33. In one embodiment of any aspect, the level and/or activity of IL-33 is increased by at least 50%, at least 60%, at least 70%, at least 80%, at least 90%, at least 99%, or more as compared to an appropriate control.

One aspect of the invention described herein provides a method to prevent damage due to a brain injury in a subject at risk of having a brain injury, comprising administering to a subject at risk of a brain injury an agent that inhibits sST2.

Another aspect of the invention described herein provides a method to prevent damage due to a brain injury in a subject at risk of having a brain injury, comprising administering to a subject at risk of a brain injury an agent that upmodulates IL-33.

In one embodiment of any aspect, the subject does not have a cardiac injury.

In one embodiment of any aspect, damage is a poor functional prognosis or hemorrhagic transformation.

One aspect of the invention described herein provides a method to prevent a poor function prognosis after a brain injury, the method comprising; (a) measuring a level of sST2 in a biological sample from a subject with a brain injury; (b) identifying a subject considered to be at risk of a poor functional prognosis when the level of sST2 in the biological sample is increased as compared to a reference level; and (c) administering to the subject identified to be at risk a prophylactic treatment.

In one embodiment of any aspect, the level of sST2 is increased at least 2-fold, at least 3-fold, at least 4-fold, at least 5-fold, at least 6-fold, at least 7-fold, at least 8-fold, at least 9-fold, at least 10-fold, or more as compared to a reference level.

One aspect of the invention described herein provides a method to prevent a poor functional prognosis after an ischemic stroke, the method comprising; (a) measuring a level of sST2 in a biological sample from a subject who has had an ischemic stroke; (b) identifying a subject considered to be at risk of a poor functional prognosis when the level of sST2 in the biological sample is high; and (c) administering to the subject identified to be at risk a prophylactic treatment. In one embodiment, the level of sST2 in the biological sample is equal to or greater than 85 ng/mL.

One aspect of the invention described herein provides a method to prevent a poor functional prognosis after a subarachnoid hemorrhage, the method comprising; (a) measuring a level of sST2 in a biological sample from a subject who has had a subarachnoid hemorrhage; (b) identifying a subject considered to be at risk of a poor functional prognosis when the level of sST2 in the biological sample is high; and (c) administering to the subject identified to be at risk a prophylactic treatment. In one embodiment, the level of sST2 is equal to or greater than 50 ng/mL.

One aspect of the invention described herein provides a method to prevent a hemorrhagic transformation after a brain injury, the method comprising; (a) measuring a level of sST2 in a biological sample from a subject who has had a brain injury; (b) identifying a subject considered to be at risk of a hemorrhagic transformation when the level of sST2 in the biological sample is increased as compared to a reference level; and (c) administering to the subject identified to be at risk a prophylactic treatment.

In one embodiment of any aspect, the biological sample is a whole blood sample or a plasma sample.

In one embodiment of any aspect, the biological sample is taken from the subject within 48 hours of the brain injury occurring. In one embodiment of any aspect, the biological sample is taken from the subject with within 72 hours, within 96 hours, within 120 hours, within 144 hours, within 168 hours of the brain injury occurring.

In one embodiment of any aspect, the prophylactic treatment is administration of an agent that inhibits sST2. In one embodiment of any aspect, the prophylactic treatment is administration of an agent that upmodulates IL-33. In one embodiment of any aspect, any of the agents described herein are administered with at least a second treatment.

One aspect of the invention described herein provides a composition comprising an agent that inhibits sST2 for the use in treating a subject having a brain injury.

One aspect of the invention described herein provides a composition comprising an agent that upmodulates IL-33 for the use in treating a subject having a brain injury.

One aspect of the invention described herein provides a method to identify a subject at risk of a poor functional prognosis after a brain injury, the method comprising: (a) obtaining a biological sample from a subject who has had a brain injury; and (b) measuring a level of sST2 in the biological sample; wherein the subject is considered at risk of a poor functional outcome when the level of sST2 in the biological sample is increased as compared to the reference level.

One aspect of the invention described herein provides a method to identify a subject at risk of a poor functional prognosis after an ischemic stroke, the method comprising: (a) obtaining a biological sample from a subject who has had an ischemic stroke; and (b) measuring a level of sST2 in the biological sample; wherein the subject is considered at risk of a poor functional prognosis when the level of sST2 is equal to or greater than 85 ng/mL.

One aspect of the invention described herein provides a method to identify a subject at risk of a poor functional prognosis after a subarachnoid hemorrhage, the method comprising: (a) obtaining a biological sample from a subject who has had a subarachnoid hemorrhage; and (b) measuring a level of sST2 in the biological sample; wherein the subject is considered at risk of poor functional prognosis when the level of sST2 is equal to or greater than 50 ng/mL.

One aspect of the invention described herein provides a method to identify a subject at risk of a hemorrhagic transformation following a brain injury, the method comprising; (a) obtaining a biological sample from a patient who has had a brain injury; and (b) measuring a level of sST2 in the biological sample; wherein the subject is considered at risk of a hemorrhagic transformation when the level of sST2 in the biological sample is increased as compared to the reference level.

In one embodiment of any aspect, the method further comprises administering to a subject at risk of a poor function prognosis any of the compositions described herein.

In one embodiment of any aspect, the method further comprises administering to a subject at risk of a poor function prognosis a prophylactic treatment.

One aspect of the invention described herein provides a kit for detecting levels of sST2 after a brain injury, comprising: (a) an antibody that binds to human sST2; and (b) instructions for detecting levels of sST2 in a biological sample.

In one embodiment of any aspect, the kit further comprises a device to collect a biological sample.

In one embodiment of any aspect, the kit further comprises a reference level.

One aspect of the invention described herein provides a kit for detecting levels of sST2 after an ischemic stroke, comprising: (a) an antibody that binds to human sST2; and (b) instructions for detecting levels of sST2 in a biological sample.

In one embodiment of any aspect, the instructions indicate that a person is at risk of a poor functional prognosis if the levels of sST2 in the biological sample are equal to or greater than 85 ng/mL.

One aspect of the invention described herein provides a kit for detecting levels of sST2 after a subarachnoid hemorrhage, comprising: (a) an anti-body that binds to human sST2; and (b) instructions for detecting levels of sST2 in a biological sample.

In one embodiment of any aspect, instructions indicate that a person is at risk of a poor functional prognosis if the levels of sST2 in the biological sample are equal to or greater than 50 ng/mL.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with the brain injury (e.g., an inschemic stroked). The term “treating” includes reducing or alleviating at least one adverse effect or symptom of a condition associated with a brain injury. Treatment is generally “effective” if one or more symptoms or clinical markers are reduced. Alternatively, treatment is “effective” if the progression of a condition associated with the brain injury is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of the condition associated with the brain injury, stabilized (i.e., not worsening) state of the condition, delay or slowing of the progression of the condition, amelioration or palliation of the condition, remission (whether partial or total), and/or decreased mortality, whether detectable or undetectable. The term “treatment” of a condition associated with the brain injury also includes providing relief from the symptoms or side-effects of the disease (including palliative treatment).

Methods and compositions described herein are directed at preventing a poor functional outcome following a brain injury. As used herein, the term “prevent” or “preventing” refers to the prevention of at least one symptom associated with damage (e.g., caused by a brain injury, or complete prevention of damage onset and/or symptoms, or the lessening of the severity of damage and/or damage symptoms in a subject, and/or delaying one or more symptoms of damage, and/or delaying the onset of damage and/or damage symptoms following a brain injury.

As used herein, the term “administering,” refers to the placement of a therapeutic (e.g, an agent that inhibits sST2, or an agent that upmodulates IL-33) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., ischemic stroke. A subject can be male or female.

A subject can be one who has been previously diagnosed with or identified as suffering from or having a brain injury (e.g., ischemic stroke or subarachnoid hemorrhage) or one or more complications related to such a brain injury, and optionally, have already undergone treatment for the brain injury, or a previous brain injury. Alternatively, a subject can also be one who has not been previously diagnosed as having a brain injury (e.g., ischemic stroke or subarachnoid hemorrhage) or related complications. For example, a subject can be one who exhibits one or more risk factors for a brain injury (e.g., high blood pressure) or one or more complications related to a brain injury or a subject who does not exhibit risk factors.

As used herein, an “brain injury” refers to an insult to any region or portion of the brain. A brain injury can be traumatic, e.g., caused by an external physical force, or non-traumatic, e.g., caused by non-external factors (e.g, a stroke, an infection, an aneurysm, or a hemorrhage). As used herein, “damage” refers to the negative effects caused by a brain injury. The “damage” can be permanent or reversible, can vary in severity, and can be localized or diffuse in the brain. Exemplary damage caused by a brain injury include, but are not limited to, damage to the skull, vascular damage, hemorrhage, hemorrhagic transformation, secondary stroke, formation of blood clots, hypoxia, and swelling of the brain. In one embodiment, methods described herein prevent additional damage following a brain injury. As used herein, “additional damage” refers to secondary damage resulting from the initial brain injury.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit sST2, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, an agent is a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

Methods and compositions described herein require that the levels and/or activity of sST2 are inhibited. As used herein, “soluble suppression of tumorigenicity 2 (sST2)” refers to a member of the Toll-like/Interleukin (IL)-1 receptor family. sST2, also known as interleukin 1 receptor like 1 (ILRL1) is a receptor for IL-33. Sequences for IL1RL1 are known for a number of species, e.g., human IL1RL1 (NCBI Gene ID: 9173) polypeptide (e.g., NCBI Ref Seq NP_001269337.1) and mRNA (e.g., NCBI Ref Seq NM_001282408.1). sST2 can refer to human sST2, including naturally occurring variants, molecules, and alleles thereof. sST2 refers to the mammalian sST2 of, e.g., mouse, rat, rabbit, dog, cat, cow, horse, pig, and the like. The nucleic sequence of SEQ ID NO:1 comprises the nucleic sequence which encodes human IL1RL1 (also known as sST2).

Methods and compositions described herein require that the levels and/or activity of IL-33 are upmodulated. As used herein, “Interleukin-33 (IL-33)” refers to a cytokine that binds receptor IL1RL/ST2 receptor that is expressed on Th2 cells, mast cells, basophils, eosinophils, and natural killer cells. IL-33 sequences are known for a number of species, e.g., human IL-33 (NCBI Gene ID: 90865) polypeptide (e.g., NCBI Ref Seq NP_001186569.1) and mRNA (e.g., NCBI Ref Seq NM_001199640.1). IL-33 can refer to the human equivalent of IL-33, including naturally occurring variants, molecules, and alleles thereof. IL-33 refers to the mammalian IL-33 of, e.g., mouse, rat, rabbit, dog, cat, cow, horse, pig, and the like. The nucleic sequence of SEQ ID NO:2 comprises the nucleic sequence which encodes human IL-33.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition (e.g., no detectable level and/or activity of sST2 using assays described herein) as compared to an appropriate control.

The terms “upmodulate”, “increase”, “enhance”, or “activate” are all used herein to mean an increase by a reproducible statistically significant amount. In some embodiments, the terms “upmodulate”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant (e.g., p>0.5) increase in such level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a subject prior to a brain injury (e.g., an ischemic stroke), or a biological sample that has not been contacted with an agent disclosed herein).

As used herein, an “appropriate control” refers to an untreated, otherwise identical cell or population (e.g., a biological sample from a subject who does not have a brain injury, a subject who was not administered an agent described herein, or was administered by only a subset of agents described herein, as compared to a non-control cell, or a subject).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1D present exemplary findings that indicate elevated sST2 is associated with poor outcome (mRS 3-6) and mortality. (FIG. 1A) Median sST2 levels are higher in patients with poor outcome (38.2 ng/mL [IQR 28.6-80.0 ng/mL]) as compared to good outcome (32.6 ng/mL [IQR 23.6-45.2 ng/mL]). Wilcoxon Test, ***, p<0.0001. Box plot represents the median and IQR, whiskers the 10th and 90th percentile. (FIG. 1B) The proportion of patients with poor outcome is greater by sST2 tertile. The percentage is shown above each bar, which represents the event rate (%). Chi-squared test, ***, p=0.0003. (FIG. 1C) Median sST2 levels are higher in patients who died (51.0 ng/mL [IQR 33.4-70.3 ng/mL]) relative to survivors (33.7 ng/mL [24.1-46.23 ng/mL]). Wilcoxon Test, ***, p<0.0001. (FIG. 1D) The number of patients who died increase by sST2 tertile. The percentage is shown above each bar. Chi-squared test, ***, p<0.0001.

FIG. 2 presents exemplary findings that show the Kaplan-Meier survival curve for sST2 tertiles. The solid line represents the first tertile (sST2<28.7 ng/mL), the dashed line represents the second tertile (sST2 28.7-44.2 ng/mL) and the dotted line represents the third tertile (sST2>44.6 ng/mL), p<0.0001.

FIGS. 3A-3D present exemplary findings that indicate elevated sST2 is associated with hemorrhagic transformation after stroke. (FIG. 3A) Median sST2 levels in patients without FIT (42.1 ng/mL [IQR 31.2-57.4 ng/mL]) and patients with 1-IT (49.7 ng/mL [IQR 35.3-70.3 ng/mL]). Wilcoxon Test, *, p=0.030. Box plot represents the median and IQR, whiskers the 10^{th} and 90^{th} percentile. (FIG. 3B) Rate of HT by sST2 tertiles. The percentage is shown above each bar, which represents the event rate (%). Chi-squared test, *, p=0.049. (FIG. 3C) Median sST2 levels in patients with HT≥HI2 (54.9 ng/mL [IQR 39.8-71.5 ng/mL]) versus those without (42.1 ng/mL [30.3-57.5 ng/mL]). Wilcoxon Test, **, p=0.008. (FIG. 3D) Rate of HT≥HI2 by sST2 tertiles. The percentage is shown above each bar. Chi-squared test, **, p=0.007.

FIG. 4 presents exemplary findings that indicate elevated sST2 is associated with neurological deaths. “Neuro” indicates the number of neurological deaths in each sST2 tertile. “Other*” indicates the number of cardiac, non-neurological/non-cardiac, and unknown deaths in each sST2 tertile. The number of neurological deaths was significantly higher in the highest sST2 tertile compared with the lower sST2 tertiles, p=0.033.

FIGS. 5A-5F present exemplary findings that indicate sST2 tertiles, quartiles and quintiles were significantly associated with poor outcome and mortality. The highest tertile, quartile and quintile were associated with poor outcome ((FIG. 5A), p=0.0003; (FIG. 5C), p<0.0001; and (FIG. 5E), p=0.0005, respectively) and mortality ((FIG. 5B), p<0.0001; (FIG. 5D), p<0.0001; and (FIG. 5F) p<0.0001, respectively).

FIGS. 6A-6D present exemplary findings that indicate sST2 is associated with poor outcome and mortality regardless of tPA treatment status. Box plot represents the median and IQR, whiskers the 10th and 90th percentile. (FIG. 6A) Median sST2 levels are higher in patients with poor outcome (37.4 ng/mL [IQR 27.9-54.5 mg/mL]) as compared to good outcome (33.9 mg/mL [IQR 23.5-45.1 ng/ml]) in patients that did not receive tPA. Wilcoxon Test (p=0.041). (FIG. 6B) Median sST2 levels are higher in patients with poor outcome (39.8 ng/mL [IQR 31.0-59.3 ng/mL]) as compared to good outcome (31.1 ng/mL [IQR 24.0-45.7 ng/mL]) in patients that received tPA. Wilcoxon Test (p=0.0001). (FIG. 6C) Median sST2 levels are higher in patients who died (41.6 mg/mL [IQR 27.7-70.1 ng/mL]) as compared who lived (34.3 ng/mL [IQR 23.8-45.7 ng/mL]) in patients that did not receive tPA. Wilcoxon Test (p=0.012). (FIG. 6D) Median sST2 levels are higher in patients who died (55.0 ng/mL [IQR 35.7-78.6 ng/mL]) as compared who lived (33.2 ng/mL [IQR 25.7-46.4 ng/mL]) in patients that received tPA. Wilcoxon Test (p<0.0001).

FIGS. 7A and 7B present exemplary findings that indicate sST2 is associated with hemorrhagic transformation regardless of tPA treatment status. Box plot represents the median and IQR whiskers the 10th and 90th percentile. (FIG. 7A) Median sST2 levels are significantly higher in patients who developed hemorrhagic transformation compared to those that did not develop hemorrhagic transformation in patients that did not receive tPA. Wilcoxon Test (p=0.041). (FIG. 7B) Median sST2 levels are significantly higher in patients who developed hemorrhagic transformation compared to those that did not develop hemorrhagic transformation in patients that received tPA. Wilcoxon Test (p=0.014).

FIGS. 8A and 8B presents exemplary findings that indicate that levels of sST2 are increased following a subarachnoid hemorrhage. A time course following a subarachnoid hemorrhage. Samples are taken from a subject following a subarachnoid hemorrhage. (FIG. 8A) Levels of sST2 in plasma following a subarachnoid hemorrhage. (FIG. 8B) Levels of sST2 in serebral spinal fluid (CSF) following a subarachnoid hemorrhage.

FIG. 9 presents exemplary findings that indicate the levels of sST2 is an indicator for the survival rate.

FIGS. 10A and 10B presents exemplary findings that indicate the levels of sST2 predicts future DCL (FIG. 9A) Quantification of plasma sST2 versus DCI. p=0.0026. (FIG. 9B) A correlation of true positive sensitivity versus 1-specificity false positive. ROC threshold=sST2>77 ng/mL. sST2 levels are measured at 3.5 following brain injury (e.g., SAH).

FIGS. 11A and 11B presents exemplary findings that indicate the levels of sST2 predicts poor outcome and DO. (FIG. 10A) Levels of sST2 in good outcome versus bad outcome at a given time after brain injury (e.g., SAH). (FIG. 10B) Levels of sST2 in DCI versus no DCI at a given time after brain injury (e.g., SAH).

### DETAILED DESCRIPTION

The invention described herein is related in part to the discovery that sST2 functions as a biomarker for a poor functional outcome and mortality following an ischemic stroke and aneurysmal subarachnoid hemorrhage. Data presented herein indicates that increased levels of sST2 in a plasma sample taken from a subject 1 day and 5 days following a brain injury (e.g., an ischemic stroke, and subarachnoid hemorrhage) was independently associated with poor outcome and mortality within 90 days of the initial brain injury.

A soluble form of ST2 (sST2) is secreted into the circulation and functions as a decoy receptor that sequesters IL-33 and blocks membrane bound signaling¹¹. This inhibition tips the immune response away from the Th-2 inflammatory response toward a Th1 pro-inflammatory response. This pro-inflammatory signal may increase the probability of a poor functional prognosis, as well as increase the damage caused by an ischemic stroke and/or a subarachnoid hemorrhage. Moreover, increased levels of sST2 following an ischemic stroke functions as a biomarker that indicates the likelihood of a subject having a hemorrhagic transformation following an ischemic stroke.

ST2 is a member of the Toll-like/Interleukin-1 receptor family that is expressed on macrophages and T helper (Th) cells⁶. Circulating levels of sST2 are associated with adverse outcome and mortality in patients with chronic heart failure (CHF) and myocardial infarction (MI).^{7,8,9} Recently, higher sST2 has been associated with an elevated risk of incident stroke.¹⁰ Administration of interleukin-33 (IL-33) can ameliorate the pro-inflammatory response, reduce ischemic damage, and improve neurological function.¹¹ The functional ligand for ST2, IL-33, was first identified when its expression was induced in canine endothelial cells in response to subarachnoid hemorrhage⁷. ST2/IL-33 binding stimulates Th cells to adopt an anti-inflammatory Th2 cellular identity⁸⁻⁹. Accordingly, IL-33 has been shown to reduce tissue injury and improve outcome in a murine model of stroke¹⁰.

It is specifically contemplated herein that administering an agent that inhibits sST2, or an agent that upmodulates IL-33, will overcome the pro-inflammatory response caused by the brain injury, and reverse the damage caused, and/or prevent the onset of damage caused by the brain injury.

Upmodulate or upmodulation refers to increasing the function of the protein (e.g., IL-33C). This can be accomplished by directly increasing or activating the production of IL-33 itself in the cell (e.g., by increasing gene expression or protein synthesis), or alternatively by increasing its function/activity. Function/activity of IL-33 can be increased, for example by directly activating protein itself. As such, an agent useful in the present invention for upmodulation is one that increases or activates the gene expression or protein synthesis of IL-33. Upmodulation of IL-33 can also be accomplished by increasing or activation of an upstream factor that induces or positively regulates gene expression or function/activity of IL-33.

Brain Injury

Various aspects described herein relate to a method of increase clinical outcome following a brain injury, prevent damage following a brain injury, or identify a subject at risk of a poor functional prognosis after a brain injury. In some embodiments, a “brain injury” refers to a frontal lobe injury, a broca area injury, a motor strip injury, a sensory strip injury, a parietal lobe injury, a Wernicke area injury, a temporal lobe injury, an occipital lobe injury, a cerebellum injury, or the brainstem injury. A brain injury can result in damage to the tissue, and/or the blood vessels within the tissue. An acquired brain injury refers to a brain injury caused by effects acquired after birth, e.g., not due to genetic or congenital disorders. An acquired brain injury is classified as being a traumatic brain injury or a non-traumatic brain injury based on the cause of the injury.

A traumatic brain injury is the most common type of brain injury, affecting as many as 1.7 million people annually, and resulting in 52,000 deaths annually. A traumatic brain injury refers to an injury caused by a physical blow, e.g., to the head or body, that results in damage to the brain tissue, vascular system, or surrounding tissue or bone (e.g., the skull). The specific prognosis of a patient who has had a traumatic brain injury is dependent on the location of the impact, the severity of the impact, overall brain health prior to the impact, and age.

Exemplary traumatic brain injuries include, but are not limited to, coup-contrecoup brain injury, concussion, diffuse axonal injury, second impact syndrome, brain contusion, shaken baby syndrome, and penetrating injury. The most common type of traumatic brain injury is a concussion.

A non-traumatic brain injury can be caused by either internal or external sources, e.g., a cerebral stroke, brain tumors, an infection, poisoning, hypoxia (e.g., a lack of oxygen to the brain), ischemia, encephalophathy, subarachnoid hemmorhage or substance abuse. Exemplary cerebral strokes include, but are not limited to, acute ischemic stroke, transient ischemic attack, and a hemorrhagic stroke.

An ischemic stroke occurs when a blood clot enters an artery in the brain causing a blockage that results in a lack of oxygen and nutrients to the brain. Ischemic strokes can be classified into two types: thrombotic stroke and embolic stroke. A “thrombotic stroke” refers to a stroke caused by diseased or damaged cerebral arteries become blocked by the formation of a blood clot within the brain. Cerebral thrombosis can also be divided into an additional two categories that correlate to the location of the blockage within the brain: large-vessel thrombosis and small-vessel thrombosis. Large-vessel thrombosis is the term used when the blockage is in one of the brain's larger blood-supplying arteries such as the carotid or middle cerebral, while small-vessel thrombosis involves one (or more) of the brain's smaller, yet deeper, penetrating arteries. This latter type of stroke is also called a lacuner stroke.

An “embolic stroke” refers to a stroked caused by a clot within an artery, however the clot (e.g., the emboli) forms somewhere other than in the brain itself. Often from the heart, these emboli will travel in the bloodstream until they become lodged and cannot travel any farther. This restricts the flow of blood to a region of the brain and can results in near-immediate physical and neurological deficits.

Subjects at risk of having an ischemic stroke are those with narrowed arteries, high cholesterol, a clotting disorder, and/or high blood pressure.

A hemorrhagic stroke refers to a stroke caused by a bleed in the brain resulting from a ruptured aneurysm or a weak blood vessel. Hemorrhagic strokes include intracerebral hemorrhagic strokes and subarachnoid hemorrhagic strokes.

A subarachnoid hemorrhage is a type of stroke caused by bleeding into the space surrounding the brain. Subarachnoid hemorrhage can be caused by a ruptured aneurysm, arteriovenous malformation, or head injury. The subarachnoid space is the area between the brain and the skull normally filled with cerebrospinal fluid (CSF). When blood is released into the subarachnoid space, it irritates the lining of the brain, increases pressure on the brain, and damages brain cells.

In one embodiment, the brain injury is a focal brain injury. As used herein, a “focal brain injury” refers to an injury to an isolated part of the brain (e.g., only the frontal lobe is effected). Focal brain injuries are often caused by a mechanical force (e.g., a penetrating wound or concussion), and the damage is easily identified by standard diagnostics (e.g., non-invasive imaging). Exemplary focal brain injuries include, but are not limited to, intraventricular hemorrhage, a subdural hemorrhage, an intracerebral hemorrhage, a cerebral contusion, a cerebral laceration, and an epidural hemorrhage.

In one embodiment, the brain injury is a diffuse brain injury. As used herein, “diffuse brain injury” refers to an injury that multiple areas of the brain, or the entire area of the brain. Exemplary diffuse brain injuries include, but are not limited to hypoxia, hypoxia caused by a stroke, an infection (e.g., a meningitis infection), or damage to blood vessels. Diffuse brain injuries are often harder to identify and define.

Methods and compositions described herein provide a method of improving clinical outcome following a brain injury, the method comprising administering to a subject who has had a brain injury an agent that inhibits sST2. In one embodiment, the clinical outcome is improved at least 10%, at least 20%, at least 30%, at least 40%, at least 50%, at least 60%, at least 70%, at least 80%, at least 90%, at least 95%, at least 99%, or more as compared to an appropriate control. Clinical outcome can be measured by assessing the degree of damage caused by the brain injury, the additional damage present following the brain injury, the functional outcome, and the ability for the subject to recover from the brain injury. As used herein, an appropriate control refers to a subject who has had a similar brain injury but was not administered the agent that inhibits sST2.

Also provided herein is a method of improving clinical outcome, the method comprising administering to a subject who has had a brain injury an agent that upmodulated IL-33. In one embodiment, the clinical outcome is improved at least 10%, at least 20%, at least 30%, at least 40%, at least 50%, at least 60%, at least 70%, at least 80%, at least 90%, at least 95%, at least 99%, or more as compared to an appropriate control. Clinical outcome can be measured by assessing the degree of damage caused by the brain injury, the additional damage present following the brain injury, the functional outcome, and the ability for the subject to recover from the brain injury (e.g., using the methods described below or known in the art). As used herein, an appropriate control refers to a subject who has had a similar brain injury but was not administered the agent that upmodulated IL-33.

Diagnosing a Brain Injury

Various techniques known in the art are used to diagnose the type of brain injury and the severity of a brain injury. The diagnostic techniques described herein are used to determine which treatment should be given to a subject following a brain injury.

Symptoms of a mild traumatic brain injury include, but are not limited to, brief loss of consciousness (e.g., seconds to a few minutes), headache, fatigue, vomiting, problems with speech, dizziness, and/or loss of balance. Symptoms of a moderate to severe traumatic brain injury include, but are not limited to, loss of consciousness (e.g., a few minutes to hours), severe headache, convulsions and seizures, clear fluid draining from ears and/or nose, dilation in one or both eyes, numbness or weakness in fingers or toes, and/or loss of coordination.

The Glascow Coma Scale (GCS) is used to assess the functionality of a subject who has had or is thought to have a brain injury in three areas: 1) speech, 2) vision, and 3) mobility. A skilled practitioner will assess whether a subject speaks normally, in a manner that does not make sense, or if the subject is non-verbal. The subject is assessed for their ability to open their eyes and follow the movement of an object in front of them. Finally, the subject is assessed for the ability to move their arms, ranging from “freely moving” to “unable to move” even in response to a painful stimulus. Points are given when a subject can perform a task. A score of 13 or higher indicates a mild brain injury, 9-12 indicates a moderate brain injury, and 8 or below indicates a severe brain injury. There is no correlation between GCS score and short- or long-term recovery following a brain injury.

Protocols for assessing certain brain injuries, e.g., concussions, include assessing movement and reactivity of the eyes, testing the sense of smell in each nostril, testing the ability for a subject to whistle, smile evenly, and clench their teeth, testing for equal hearing in both ears, testing for unilateral or bilateral motor weakness, cognitive function, and determine is muscle tremors are present.

Non-invasive imaging is most commonly used to assess a subject who is thought to have had a brain injury. A computerized topography (CT) scan is commonly performed on a subject with a suspected brain injury to assess gross damages to the brain. A CT scan can readily show, e.g., areas of abnormalities in the brain, areas of reduced blood flow (e.g., a stroke), or a ruptured blood vessel in the brain. CT scans are a primary method of determining whether a stroke is ischemic or hemorrhagic. Magnetic resonance imaging (MRI) is used to obtain more fine detail of the, e.g., brain tissue; an MRI provides higher resolution of the brain tissue as compared to CT scan. MRIs can be performed with a contrast dye to allow for precise imaging of the blood vessels, e.g., a magnetic resonance angiography (MRA).

A stroke is diagnosed by a blockage in the blood vessels of the brain and the lack of blood flowing to a region of the brain. CT scans and MRIs are used to diagnose a stroke and identify the location of the stroke. Other symptoms of a stroke, e.g., dropping of one side of the face, inability to speak properly, or weakness on one side of the body, are identified by a neurological exam.

A subarachnoid hemorrhage is diagnosed by a CT scan or MRI to identify abnormal regions of the subarachnoid space (e.g., filled with blood), or defects in the blood vessels. Additionally, a lumbar puncture can be performed to assess whether blood is present in the CSF.

Treating a Brain Injury

The treatment for a brain injury is dependent on the type of brain injury (e.g., traumatic brain injury vs. a non-traumatic brain injury), the severity of the brain injury (e.g., mild, moderate, or severe), and the risk of additional damage following the brain injury (e.g., age, overall health, and history of brain injuries). Treatments can range from rest, medication, to surgery, and can be administered or performed by a skilled practitioner.

Closely monitored rest (e.g., limited physical and cognitive activities) can be prescribed to a subject having a mild brain injury, with no further medical intervention.

Medication may be used to treat subjects who have had a brain injury. For example, a subject presenting with mild swelling of the brain can be prescribed a diuretic (e.g., Mannitol) to increase urine production and reduce the levels of excess fluid in the body. Anti-seizure medications can be prescribed prophylactically to a subject having a moderate to severe brain injury. The anti-seizure medication is used to prevent further brain injuries in the weeks following an initial brain injury.

A subject having severe brain damage can be placed in a medical-induced coma. A brain that has, e.g., compressed blood vessels due to swelling and/or increased pressure on the brain, is unable to supply the proper levels of blood and oxygen the brain. A comatose brain, however, requires less oxygen to function. Thus, a medical-induced coma allows the brain to heal while receiving less oxygen.

Surgical procedures are used to minimize additional damage to the brain following a brain injury. Surgery following a brain injury is often emergent. Bleeding outside (e.g., the subarachnoid space) or within the brain can result in a collection of hematomas that increase the pressure in the brain. Surgery is used to remove hematomas (e.g., clotted blood) from the brain tissue. During a traumatic brain injury, the skull can become fractured. Surgical procedures are used to repair damage to the skull (e.g., severe skull fractures) or to remove pieces of the skull in the brain. Surgery can also be used to stop a hemorrhage (e.g., a brain bleed) that caused the brain injury or is present after a brain injury. Certain brain injuries, e.g., subarachnoid hemorrhages, can be caused be a ruptured aneurysm. Surgical procedures are used to stop bleeding from a ruptured aneurysm (e.g., surgical clipping, or endovascular coiling).

Swelling of the brain occurs following certain brain injuries, dangerously increasing the cranial pressure. Decompressive craniectomy is a surgical procedure that removes a section of the skull to allow a swollen brain room to expand, relieving cranial pressure following a brain injury.

Rehabilitation is used to retain the brain's pathways to improve mental and physical functionality after a brain injury. Rehabilitation is often prescribed to subjects who have lost the ability to perform tasks they were able to easily do prior to the brain injury, e.g., speak properly, eat, walk, problem solve, recall memories, etc. Speech therapy and occupational therapy may also be prescribed.

Cognitive therapy is prescribed to subjects who exhibit behavioral changes following a brain injury. For example, a subject may exhibit changes in their mood (e.g., anger, anxiety, apathy, or depression), or their behavior (e.g., abnormal laughing and crying, aggression, impulsivity, irritability, lack of restraint, or persistent repetition of words). Anger management can also be prescribed.

The risk of a second injury following a non-traumatic brain injury is greatly increased. Medication is prescribed to a subject following a non-traumatic brain injury to prevent a second injury. For example, a subject can be prescribed medication to prevent high blood pressure (e.g., a thiazide diuretic, a potassium-sparing diuretic, a loop diuretic, or a combination diuretic), to prevent atrial fibrillation (e.g., aspirin, or an anti-coagulant), or a medication that prevents the formation of blood clots (e.g., antiplatelet agents, or anticoagulants, e.g., warfarin) to prevent a second ischemic stroke following a first ischemic stroke.

Healthy lifestyle changes are recommended to a subject who has had a brain injury (e.g., a non-traumatic brain injury). These lifestyle changes include managing blood pressure, maintaining a healthy weight, stopping smoking, and increasing physical activity.

The proinflammatory response activated by the expression of sST2, and the resulting decrease in IL-33 signaling can increase the severity of the brain injury and resulting damage. Thus, in various aspects of the invention described herein, a subject is administered an agent that inhibits sST2, or an agent that upmodulates IL-33 following a brain injury. In various embodiments, the agent is administered in combination with at least a second treatment as described herein for a brain injury.

Any of the treatments described herein can be prophylactically administered to a subject who has had a brain injury and has been identified as being at risk of a poor functional outcome following a brain injury. In various embodiments, a subject is identified as being at risk of a poor functional outcome and is administered a prophylactic treatment to prevent the poor functional outcome.

Poor Functional Outcome Following a Brain Injury

Following a brain injury, the subject's functional outcome can be dependent on the severity of the brain injury, the type of injury, and the overall health of the subject prior to the brain injury. As used herein, “functional outcome” refers to the subject's capacity heal following a brain injury. The functional outcome refers to the severity of the damage caused to the brain, the likelihood of acquiring additional damage to the brain following the initial brain injury, and/or the likelihood of dying as a result of the brain injury within 90 days of the injury occurring. As used herein, “acquiring additional damage” refers to any additional damage acquired following a brain injury, e.g., a hemorrhagic transformation following a stroke, delayed cerebral ischemia, cerebral vasospasm, or cortical spreading depression.

As used herein, a “poor functional outcome” refers to an outcome that suggests that the subject will not fully recover from the brain injury, will acquire more damage to the brain following a brain injury, and/or will die within 90 days of the brain injury.

The poor functional outcome can additionally indicate that the subject's will not be able to recover their motor function, including fine motor skills and gross motor skills, cognitive function, and/or language function following a brain injury. For example, a subject with a poor functional outcome is more likely to not recover a loss of speech caused by an ischemic stroke.

Preventing a Poor Functional Outcome Following a Brain Injury

One aspect of the invention described herein provides a method to prevent a poor functional outcome in a subject after a brain injury, comprising measuring the level of sST2 in a biological sample following a brain injury, comparing the level of sST2 to a reference level, and administering to a subject identified as being at risk a prophylactic treatment. A subject is identified as being at risk of a poor functional prognosis following a brain injury if the sST2 levels is increased at least 2-fold, at least 3-fold, a least 4-fold, at least 5-fold, at least 6-fold, at least 7-fold, at least 8-fold, at least 9-fold, or at least 10-fold or more as compared to a reference level. As used herein, a reference level refers to the level of sST2 in an identical biological sample taken from a healthy individual, or the individual prior to the brain injury.

One aspect of the invention described herein provides a method to prevent a poor functional outcome in a subject after an ischemic stroke, comprising measuring the level of sST2 in a biological sample following an ischemic stroke, comparing the level of sST2 to a reference level, and administering to a subject identified as being at risk a prophylactic treatment. A subject is identified as being at risk of a poor functional prognosis following an ischemic stroke if the sST2 levels is equal to or greater than 85 ng/mL.

Another aspect of the invention described herein provides a method to prevent a poor functional outcome in a subject after a subarachnoid hemorrhage, comprising measuring the level of sST2 in a biological sample following a brain injury, comparing the level of sST2 to a reference level, and administering to a subject identified as being at risk a prophylactic treatment. A subject is identified as being at risk of a poor functional prognosis following a subarachnoid hemorrhage if the sST2 levels is equal to or greater than 50 ng/mL.

In one embodiment, the biological sample is a blood or plasma sample. In one embodiment, the biological sample is taken from the subject within 48 hours of the brain injury. In another embodiment, the biological sample is taken from the subject within 1 hour, within 6 hours, within 12 hours, within 18 hours, within 24 hours, within 30 hours, within 36 hours, within 42 hours, within 72 hours, within 96 hours, within 144 hours, within 168 hours, or more of the brain injury. A biological sample can be taken from the subject using standard techniques, e.g., drawing blood from a vein or artery.

Identifying a Subject at Risk of a Poor Functional Prognosis

One aspect of the invention described herein provides a method to identify a subject at risk of a poor functional outcome after a brain injury, comprising obtaining a biological sample from a subject, measuring the level of sST2 is a biological sample following a brain injury, and comparing the level of sST2 to a reference level. A subject is identified as being at risk of a poor functional prognosis following a brain injury if the sST2 levels is increased at least 2-fold, at least 3-fold, a least 4-fold, at least 5-fold, at least 6-fold, at least 7-fold, at least 8-fold, at least 9-fold, or at least 10-fold or more as compared to a reference level. As used herein, a reference level refers to the level of sST2 in an identical biological sample taken from a healthy individual, or the individual prior to the brain injury.

One aspect of the invention described herein provides a method to identify a subject at risk of a poor functional outcome after an ischemic stroke, comprising obtaining a biological sample from a subject and measuring the level of sST2 is a biological sample following an ischemic stroke. A subject is identified as being at risk of a poor functional prognosis following an ischemic stroke if the sST2 levels is equal to or greater than 85 ng/mL.

Another aspect of the invention described herein provides a method to identify a subject at risk of a poor functional outcome after a subarachnoid hemorrhage, comprising obtaining a biological sample from a subject and measuring the level of sST2 is a biological sample following an ischemic stroke. A subject is identified as being at risk of a poor functional prognosis following a subarachnoid hemorrhage if the sST2 levels is equal to or greater than 50 ng/mL.

In one embodiment, the biological sample is a blood or plasma sample. In one embodiment, the biological sample is taken from the subject within 48 hours of the brain injury. In another embodiment, the biological sample is taken from the subject within 1 hour, within 6 hours, within 12 hours, within 18 hours, within 24 hours, within 30 hours, within 36 hours, within 42 hours, within 72 hours, within 96 hours, within 144 hours, within 168 hours, or more of the brain injury.

Hemorrhagic Transformation

Following a brain injury (e.g., an ischemic stroke), a subject can be at risk of developing hemorrhagic transformation. As used herein, a “hemorrhagic transformation” refers to a reperfusion of blood into the ischemic tissue at an embolic or thrombotic event. Symptoms of a hemorrhagic transformation include, but are not limited to a decrease in neurological function, head pain, loss of consciousness, dizziness, and neck rigidity. Hemorrhagic transformations are spontaneous complications of an, e.g., an ischemic stroke, or spontaneously occur after, e.g., treatment with alteplase and other intravenous tissue plasminogen activator (IV tPA) agents. Hemorrhagic transformation can be diagnosed using, e.g., non-invasive imaging (e.g., CT scan, or MRI).

One aspect of the invention described herein provides a method to prevent a hemorrhagic transformation in a subject following a brain injury (e.g., an ischemic stroke) comprising measuring the levels of sST2 in a biological sample obtained from a patient who has had a brain injury, and comparing it to a reference level, and administering to a subject at risk of a hemorrhagic transformation a prophylactic treatment. A subject is identified as being at risk of a hemorrhagic transformation if the sST2 is increased at least 2-fold, at least 3-fold, a least 4-fold, at least 5-fold, at least 6-fold, at least 7-fold, at least 8-fold, at least 9-fold, or at least 10-fold or more as compared to a reference level. As used herein, a reference level refers to the level of sST2 in an identical biological sample taken from a healthy individual, or the individual prior to the brain injury.

Another aspect of the invention described herein provides a method of identifying a subject at risk of having a hemorrhagic transformation following a brain injury (e.g., an ischemic stroke) comprising obtaining a biological sample from a subject who has or has had brain injury, and measuring the level of sST2 in the biological sample. A subject is considered at risk of having a hemorrhagic transformation if the level of sST2 in the biological sample is increased at least 2-fold, at least 3-fold, a least 4-fold, at least 5-fold, at least 6-fold, at least 7-fold, at least 8-fold, at least 9-fold, or at least 10-fold or more as compared to a reference level. As used herein, a reference level refers to the level of sST2 in an identical biological sample taken from a healthy individual, or the individual prior to the brain injury.

In one embodiment, the biological sample is a blood or plasma sample. In one embodiment, the biological sample is taken from the subject within 48 hours of the brain injury. In another embodiment, the biological sample is taken from the subject within 72 hours, within 96 hours, within 144 hours, within 168 hours, or more of the brain injury.

Preventing Damage Caused by a Potential a Brain Injury

Methods and compositions described herein are used to prevent damage following a brain injury in a patient who is at risk of having a brain injury. Subjects at risk of having a traumatic brain injury include, but are not limited to, children between the ages of newborn to 4-years old, young adults between the ages of 15-24, adults 60 and older, and males of any age group. In addition, those subjects who participate in activities that result in impact of high physical force, e.g., contact sports (e.g., football, rugby, and hockey), are at higher risk of a traumatic brain injury.

Subjects at risk of non-traumatic brain injury exhibit risk factors that include, but are not limited to, high blood pressure, diabetes, heart disease, smoking, previous non-traumatic brain injury, family history of non-traumatic brain injury, or having arteriovenous malformations.

Alternatively, a subject does not need to exhibit risk factors for a brain injury.

Administration of the agents described herein can reduce the damage caused by a brain injury by at least 10%, by at least 20%, by at least 30%, by at least 40%, by at least 50%, at least 60%, by at least 70%, by at least 80%, by at least 90%, by at least 99% as compared to an untreated subject. Alternatively, administration of the agents described herein can prevent any damage from occurring following a brain injury.

A subject can have previously had a brain injury and/or undergone treatment for a brain injury, and is at risk of another brain injury.

One aspect of the invention described herein provides a method to prevent damage caused by a brain injury in a subject at risk of a brain injury, comprising administering to a subject at risk of a brain injury an agent that inhibits sST2.

Another aspect of the invention described herein provides a method to prevent damage caused by a brain injury in a subject at risk of a brain injury, comprising administering to a subject at risk of a brain injury an agent that upmodulated IL-33.

In one embodiment, the subject does not have a cardiac injury.

Agents that Modulate sST2 and IL-33

In various embodiments, an agent that inhibits sST2 is administered to a subject having or at risk of having a brain injury. In one embodiment, the agent is a small molecule, an antibody or antibody fragment, a peptide, an antisense oligonucleotide, a genome editing system, or an RNAi.

An agent described herein targets sST2 for its inhibition. An agent is considered effective for inhibiting sST2 if, for example, upon administration, it inhibits the presence, amount, activity and/or level of sST2, respectively, in the cell.

An agent can inhibit e.g., the transcription, or the translation of sST2 in the cell. An agent can inhibit the activity or alter the activity (e.g., such that the activity no longer occurs, or occurs at a reduced rate) of sST2 in the cell (e.g., sST2's expression). In one embodiment, an agent binds to sST2 and prevents sST2 binding to IL-33. mRNA and protein levels of a given target (e.g., sST2) can be assessed using RT-PCR and western blotting, respectively. Biological assays that detect the activity of sST2 (e.g., binding to and inhibiting IL-33 function) can be used to assess if sST2's activity have been inhibited or altered. Alternatively, immunofluorescence detection using antibodies specific to sST2 in combination with cell death markers (e.g., Caspase) can be used to determine if cell death has occurred following administration of an agent.

In one embodiment, an agent inhibits the level and/or activity of sST2 by at least 2-fold, by at least 3-fold, by at least 4-fold, by at least 5-fold, by at least 6-fold, by at least 7-fold, by at least 8-fold, by at least 9-fold, by at least 10-fold or more as compared to an appropriate control, or by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of sST2 prior to administration of the agent, or the level and/or activity of sST2 in a population of cells that was not in contact with the agent.

The agent may function directly in the form in which it is administered. Alternatively, the agent can be modified or utilized intracellularly to generate an inhibitor of sST2, for e.g., introduction of a nucleic acid sequence into the cell and its transcription resulting in the production of the nucleic acid and/or protein inhibitor of sST2 within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits sST2. Methods for screening small molecules are known in the art and can be used to identify a small molecule that is selective for, e.g., inhibiting sST2, given the desired target (e.g., sST2).

In various embodiments, the agent that inhibits sST2 is an antibody or antigen-binding fragment thereof, or an antibody reagent that is specific for sST2. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent that inhibits sST2 is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is for therapeutic use.

In one embodiment, the antibody or antibody reagent binds to an amino acid sequence that corresponds to the amino acid sequence encoding sST2 (SEQ ID NO: 3).

(SEQ ID NO: 3)
MYSTVSGSEK NSKIYCPTID LYNWTAPLEW FKNCQALQGS
RYRAHKSFLV IDNVMTEDAG DYTCKFIHNE NGANYSVTAT
RSFTVKDEQG FSLFPVIGAP AQNEIKEVEI GKNANLTCSA
CFGKGTQFLA AVLWQLNGTK ITDFGEPRIQ QEEGQNQSFS
NGLACLDMVL RIADVKEEDL LLQYDCLALN LHGLRRHTVR
LSRKNPSKEC F

In another embodiment, the anti-sST2 antibody or antibody reagent binds to an amino acid sequence that comprises the sequence of SEQ ID NO: 3; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 3. In one embodiment, the anti-sST2 antibody or antibody reagent binds to an amino acid sequence that comprises the entire sequence of SEQ ID NO: 3. In another embodiment, the antibody or antibody reagent binds to an amino acid sequence that comprises a fragment of the sequence of SEQ ID NO: 3, wherein the fragment is sufficient to bind its target, e.g., sST2.

In one embodiment, the agent that inhibits sST2 is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., sST2. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits sST2 may comprise at least 5, at least 10, at least 15, at least 20, at least 25, at least 30, or more bases complementary to a portion of the coding sequence of the human sST2 gene (e.g., SEQ ID NO:1).

In one embodiment, sST2 is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In some embodiments, the agent inhibits sST2 by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. sST2. In some embodiments of any of the aspects, the agent is siRNA that inhibits sST2. In some embodiments of any of the aspects, the agent is shRNA that inhibits sST2.

One skilled in the art can design siRNA, shRNA, or miRNA to target sST2, e.g., using publically available design tools. siRNA, shRNA, or miRNA is can be obtained commercially, e.g., from Dharmacon (Layfayette, Colo.) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits sST2. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., sST2), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., sST2, for its downregulation, for example by transfecting the siRNA, shRNA, or miRNA into cells and detecting the levels of a gene (e.g., sST2) found within the cell via western-blotting.

In one embodiment, IL-33 is upmodulated by a nucleic acid encoding IL-33 expressed in the cell e.g., via a vector comprising a nucleic acid encoding IL-33. In another embodiment, a nucleic acid encoding IL-33 is expressed in the cell e.g., via expression of a nucleic acid encoding IL-33 as naked DNA. In one embodiment, the nucleic acid encoding IL-33 has a sequence corresponding to the sequence of SEQ ID NO: 2; or comprises the sequence of SEQ ID NO: 2; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 2, and having the same activity as the sequence of SEQ ID NO: 2 (e.g., induction of Th cells).

In one embodiment, an agent that upmodulates IL-33 can increase the activity and/or levels of an IL-33 activator. In another embodiment, an agent that upmodulates IL-33 can decrease the activity and/or activity of an IL-33 inhibitor.

In one embodiment, an agent upmodulates the level and/or activity of IL-33 by at least 2-fold, by at least 3-fold, by at least 4-fold, by at least 5-fold, by at least 6-fold, by at least 7-fold, by at least 8-fold, by at least 9-fold, by at least 10-fold or more as compared to an appropriate control, or by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of IL-33 prior to administration of the agent, or the level and/or activity of IL-33 in a population of cells that was not in contact with the agent.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an sST2 inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

One aspect of the invention described herein provides a composition comprising any of the agents that inhibit sST2 described herein for the use of treating a subject having a brain injury.

Another aspect of the invention described herein provides a composition comprising any of the agents that upmodulate IL-33 described herein for the use of treating a subject having a brain injury.

In one embodiment, the composition further comprises a pharmaceutically acceptable carrier. The term “pharmaceutically acceptable carrier” as used herein means a pharmaceutically acceptable material, composition or vehicle, such as a liquid or solid filler, diluent, excipient, solvent or encapsulating material, involved in carrying or transporting the active ingredient (e.g., cells) to the targeting place in the body of a subject. Each carrier must be “acceptable” in the sense of being compatible with the other ingredients of the formulation and is compatible with administration to a subject, for example a human.

Administration

In some embodiments, the methods described herein relate to treating a brain injury by administering to a subject in need thereof an agent that inhibits sST2, or an agent that upmodulated IL-33. As used herein, “treating a brain injury” refers to improving the clinical outcome of a brain injury, or preventing a poor functional prognosis following a brain injury. Subjects having, or at risk of having a brain injury can be identified by a physician using current methods of diagnosis as described above. Symptoms and/or complications of a brain injury (e.g., an ischemic stroke or subarachnoid hemorrhage), which characterize this injury and aid in diagnosis are well known in the art and include but are not limited to, severe headache, numbness in limbs, and vision loss. Tests that may aid in a diagnosis of, e.g. a brain injury, include but are not limited neurological exam, and non-invasive imaging (e.g., CT scan, or MRI scan).

The agents described herein (e.g., an agent that inhibits sST2, or an agent that upmodulates IL-33) can be administered to a subject having or at risk of having a brain injury (e.g., ischemic stroke, or subarachnoid hemorrhage) to prevent a poor functional prognosis. In some embodiments, the methods described herein comprise administering an effective amount of an agent to a subject in order to alleviate at least one symptom of a poor functional prognosis after a brain injury. As used herein, “alleviating at least one symptom of the poor functional prognosis” is ameliorating at least one condition or symptom associated with a poor functional prognosis (e.g., hemorrhage). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment, the agents described herein are administered systemically or locally (e.g., to the site of the brain injury). In one embodiment, the agents described herein are administered intravenously. The route of administration of the agents will be optimized for the type of agent being delivered (e.g., an antibody, a small molecule, an RNAi), and can be determined by a skilled practitioner.

In one embodiment, the agent is administered within 48 hours of the brain injury occurring. In another embodiment, the agent is administered within 1 hour, within 6 hours, within 12 hours, within 18 hours, within 24 hours, within 30 hours, within 36 hours, within 42 hours, within 72 hours, within 96 hours, within 120 hours, within 144 hours, within 168 hours of the brain injury occurring.

The term “effective amount” as used herein refers to the amount of an agent needed to alleviate at least one or more symptom of the poor functional prognosis. The term “therapeutically effective amount” therefore refers to an amount of an agent that is sufficient to provide a particular therapeutic effect (e.g., a prevention of poor functional prognosis) when administered to a typical subject. An effective amount as used herein, in various contexts, would also include an amount of an agent sufficient to delay the development of a poor functional prognosis, alter the course of a poor functional prognosis (e.g., hemorrhage), or reverse a symptom of the brain injury (e.g., tissue damage). Thus, it is not generally practicable to specify an exact “effective amount”. However, for any given case, an appropriate “effective amount” can be determined by one of ordinary skill in the art using only routine experimentation.

In one embodiment, the agent is administered continuously (e.g., at constant levels over a period of time). Continuous administration of an agent can be achieved, e.g., by epidermal patches, continuous release formulations, i.v. administration, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., neurological exam, among others. The dosage can be determined by a physician and adjusted, as necessary, to suit observed effects of the treatment.

Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Combination Treatments

In some embodiments, the agents described herein (e.g., an agent that inhibits sST2, or an agent that upmodulates IL-33) are administered as a monotherapy. In other embodiments, the agents described herein are administered in combination with at least one additional therapy. Additional treatments for a brain injury are described herein above.

In one embodiment, the agents are administered prior to administration of the at least one therapy. In one embodiment, the agents are administered following the administration of the at least one therapy. Administration of the agent and the at least one therapy can be done at different time points, or at substantially the same time. In certain instances, when the agent is administered with more than one therapy, the agent can be administered prior to, substantially with, or after any of the therapies. An agent that inhibits sST2 or an agent that upmodulates IL-33 can be comprised within a composition comprising an additional therapy (e.g., comprised in a composition comprising a second therapeutic treatment for a brain injury, e.g., a diuretic).

Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Efficacy

The efficacy of the agents described herein e.g., for the treatment of a brain injury (e.g., the prevention of a poor functional prognosis after a brain injury), can be determined by the skilled practitioner. However, a treatment is considered “effective treatment,” as the term is used herein, if one or more of the signs or symptoms of poor functional prognosis are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of damage to the brain tissue is halted). Methods of measuring these indicators are known to those of skill in the art and/or are described herein.

Efficacy can be assessed in animal models of a condition described herein, for example, a mouse model of cancer, a pathogenic infection model, or an appropriate animal model of autoimmune or inflammatory disease, as the case may be. When using an experimental animal model, efficacy of treatment is evidenced when a statistically significant change in a marker is observed, e.g., a slowing of weight loss, or weight gain.

Kits

One aspect of the invention described herein provides a kit for detecting levels of sST2 after a brain injury, comprising: (a) an anti-sST2 antibody that binds to human sST2; and (b) instructions for detecting levels of sST2 in a biological sample.

One aspect of the invention described herein provides a kit for detecting levels of sST2 after an ischemic stroke, comprising: (a) an anti-sST2 antibody that binds to human sST2; and (b) instructions for detecting levels of sST2 in a biological sample. In one embodiment, the instructions indicate that a person is at risk of a poor functional prognosis if the levels of sST2 in the biological sample are equal to or greater than 85 ng/mL.

One aspect of the invention described herein provides a kit for detecting levels of sST2 after a subarachnoid hemorrhage, comprising: (a) an anti-sST2 antibody that binds to human sST2; and (b) instructions for detecting levels of sST2 in a biological sample. In one embodiment, instructions indicate that a person is at risk of a poor functional prognosis if the levels of sST2 in the biological sample are equal to or greater than 50 ng/mL

In one embodiment of any aspect, the kit further comprises a device to collect a biological sample. In one embodiment, the biological sample is a blood or plasma sample. Devices useful in collecting a blood or plasma sample include, but are not limited to, a hypodermic needle, a reusable finger stick device (e.g., a lancet), or a single-use finger stick device.

In another embodiment, the biological sample is a urine or salvia sample. Devices used to collect urine or saliva samples are known in the art.

In one embodiment of any aspect, the kit further comprises a reference level. As used herein, a “reference level” refers to the level or value of sST2 derived from a biological sample obtained from a healthy individual, e.g., a subject who has not had a brain injury.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:
1) A method to improve clinical outcome after a brain injury in a subject comprising administering to the subject an agent that inhibits soluble suppression of tumorigenicity (sST2).
2) The method of paragraph 1, wherein the brain injury is a cerebral stroke, a subarachnoid hemorrhage, a focal brain injury, or a traumatic brain injury.
3) The method of paragraph 2, wherein the cerebral stroke selected from the group consisting of an acute ischemic stroke, transient ischemic attack, and a hemorrhagic stroke.
4) The method of paragraph 2, wherein the focal brain injury is selected from the group consisting of an intraventricular hemorrhage, a subdural hemorrhage, an intracerebral hemorrhage, a cerebral contusion, a cerebral laceration, and an epidural hemorrhage.
5) The method of paragraph 2, wherein the traumatic brain injury is selected from the group consisting of coup-contrecoup brain injury, concussion, diffuse axonal injury, second impact syndrome, brain contusion, shaken baby syndrome, and penetrating injury.
6) The method of paragraphs 1-3, wherein the agent is selected from the group consisting of a small molecule, an antibody, a peptide, an antisense oligonucleotide, a genome editing system, and an RNAi.
7) The method of paragraph 6, wherein the RNAi is a microRNA, an siRNA, or a shRNA.
8) The method of paragraph 6, wherein the agent is an anti-sST2 antibody for therapeutic use.
9) The method of paragraph 1, wherein inhibiting sST2 is decreasing the level and/or activity of sST2.
10) The method of paragraph 9, wherein the level and/or activity of sST2 is decreased by at least 50%, at least 60%, at least 70%, at least 80%, at least 90%, at least 99%, or more as compared to an appropriate control.
11) The method of paragraphs 1-8, wherein the agent is administered within 48 hours of the brain injury occurring.
12) The method of paragraphs 1-8, wherein the agent is administered within 72 hours, within 96 hours, within 120 hours, within 144 hours, within 168 hours of the brain injury occurring.
13) A method to improve clinical outcome after a brain injury in a subject comprising administering to the subject an agent that upmodulates interleukin-33 (IL-33).
14) The method of paragraph 11, wherein the brain injury is a cerebral stroke, a subarachnoid hemorrhage, a focal brain injury, or a traumatic brain injury.
15) The method of paragraph 12, wherein the stroke selected from the group consisting of an acute ischemic stroke, and a hemorrhagic stroke.
16) The method of paragraph 12, wherein the focal brain injury is selected from the group consisting of an intraventricular hemorrhage, a subdural hemorrhage, an intracerebral hemorrhage, a cerebral contusion, a cerebral laceration, and an epidural hemorrhage.
17) The method of paragraph 12, wherein the traumatic brain injury is selected from the group consisting of coup-contrecoup brain injury, concussion, diffuse axonal injury, second impact syndrome, brain contusion, shaken baby syndrome, and penetrating injury.
18) The method of paragraphs 11-15, wherein the agent is selected from the group consisting of a small molecule, a peptide, and an expression vector encoding IL-33.
19) The method of paragraph 13, wherein upmodulating IL-33 is increasing the level and/or activity of IL-33.
20) The method of paragraph 9, wherein the level and/or activity of IL-33 is increased by at least 50%, at least 60%, at least 70%, at least 80%, at least 90%, at least 99%, or more as compared to an appropriate control.
21) The method of paragraph 16, wherein the agent is comprised in a vector.
22) The method of paragraph 9, wherein the vector is non-integrative or integrative.
23) The method of paragraphs 11-18, wherein the agent is administered within 48 hours of the brain injury occurring.
24) The method of paragraphs 11-18, wherein the agent is administered within 72 hours, within 96 hours, within 120 hours, within 144 hours, within 168 hours of the brain injury occurring.
25) A method to prevent damage due to a brain injury in a subject at risk of having a brain injury, comprising administering to a subject at risk of a brain injury an agent that inhibits sST2.
26) A method to prevent damage due to a brain injury in a subject at risk of having a brain injury, comprising administering to a subject at risk of a brain injury an agent that upmodulates IL-33.
27) The method of paragraphs 25 and 26, wherein the subject does not have a cardiac injury.
28) The method of paragraphs 25 and 26, wherein damage is a poor functional prognosis or hemorrhagic transformation.
29) A method to prevent a poor function prognosis after a brain injury, the method comprising;
a. measuring a level of sST2 in a biological sample from a subject with a brain injury;
b. identifying a subject considered to be at risk of a poor functional prognosis when the level of sST2 in the biological sample is increased as compared to a reference level; and
c. administering to the subject identified to be at risk a prophylactic treatment.
30) The method of paragraphs 29, wherein the biological sample is a whole blood sample or a plasma sample.
31) The method of paragraphs 30 and 31, wherein the biological sample is taken from the subject within 48 hours of the brain injury occurring.
32) The method of paragraphs 30 and 31, wherein the biological sample is taken from the subject with within 72 hours, within 96 hours, within 120 hours, within 144 hours, within 168 hours of the brain injury occurring.
33) The method of paragraph 29, wherein the level of sST2 is increased at least 2-fold, at least 3-fold, at least 4-fold, at least 5-fold, at least 6-fold, at least 7-fold, at least 8-fold, at least 9-fold, at least 10-fold, or more as compared to a reference level.
34) The method of paragraph 29, wherein the prophylactic treatment is administration of an agent that inhibits sST2.
35) The method of paragraph 29, wherein the prophylactic treatment is administration of an agent that upmodulates IL-33.
36) The method of paragraph 34 and 35, wherein the agent is administered with at least a second treatment.
37) A method to prevent a poor functional prognosis after an ischemic stroke, the method comprising;
a. measuring a level of sST2 in a biological sample from a subject who has had an ischemic stroke;
b. identifying a subject considered to be at risk of a poor functional prognosis when the level of sST2 in the biological sample is high; and
c. administering to the subject identified to be at risk a prophylactic treatment.
38) The method of paragraph 37, wherein the biological sample is a whole blood sample or a plasma sample.
39) The method of paragraph 37, wherein the level of sST2 in the biological sample is equal to or greater than 85 ng/mL.
40) The method of paragraphs 37-39, wherein the biological sample is taken from the subject within 48 hours of the ischemic stroke occurring.
41) The method of paragraphs 37-39, wherein the biological sample is taken from the subject with within 72 hours, within 96 hours, within 120 hours, within 144 hours, within 168 hours of the ischemic stroke occurring.
42) The method of paragraph 37, wherein the prophylactic treatment is administration of an agent that inhibits sST2.
43) The method of paragraph 37, wherein the prophylactic treatment is administration of an agent that upmodulates IL-33.
44) The method of paragraph 42 and 43, wherein the agent is administered with at least a second treatment.
45) A method to prevent a poor functional prognosis after a subarachnoid hemorrhage, the method comprising;
a. measuring a level of sST2 in a biological sample from a subject who has had a subarachnoid hemorrhage;
b. identifying a subject considered to be at risk of a poor functional prognosis when the level of sST2 in the biological sample is high; and
c. administering to the subject identified to be at risk a prophylactic treatment.
46) The method of paragraph 45, wherein the biological sample is a whole blood sample or a plasma sample.
47) The method of paragraph 45, wherein the level of sST2 is equal to or greater than 50 ng/mL.
48) The method of paragraphs 45-47, wherein the biological sample is taken from the subject within 48 hours of the subarachnoid hemorrhage occurring.
49) The method of paragraphs 45-47, wherein the biological sample is taken from the subject with within 72 hours, within 96 hours, within 120 hours, within 144 hours, within 168 hours of the subarachnoid hemorrhage occurring.
50) The method of paragraph 45, wherein the prophylactic treatment is administration of an agent that inhibits sST2.
51) The method of paragraph 45, wherein the prophylactic treatment is administration of an agent that upmodulates IL-33.
52) The method of paragraph 50 and 51, wherein the agent is administered with at least a second treatment.
53) A method to prevent a hemorrhagic transformation after a brain injury, the method comprising;
a. measuring a level of sST2 in a biological sample from a subject who has had a brain injury;
b. identifying a subject considered to be at risk of a hemorrhagic transformation when the level of sST2 in the biological sample is increased as compared to a reference level; and
c. administering to the subject identified to be at risk a prophylactic treatment.
54) The method of paragraphs 53, wherein the biological sample is a whole blood sample or a plasma sample.
55) The method of paragraphs 53 and 54, wherein the biological sample is taken from the subject within 48 hours of the brain injury occurring.
56) The method of paragraphs 53 and 54, wherein the biological sample is taken from the subject with within 72 hours, within 96 hours, within 120 hours, within 144 hours, within 168 hours of the brain injury occurring.
57) The method of paragraph 53, wherein the level of sST2 is increased at least 2-fold, at least 3-fold, at least 4-fold, at least 5-fold, at least 6-fold, at least 7-fold, at least 8-fold, at least 9-fold, at least 10-fold, or more as compared to a reference level.
58) The method of paragraph 53, wherein the prophylactic treatment is administration of an agent that inhibits sST2.
59) The method of paragraph 53, wherein the prophylactic treatment is administration of an agent that upmodulates IL-33.
60) The method of paragraph 58 and 59, wherein the agent is administered with at least a second treatment.
61) A composition comprising an agent that inhibits sST2 for the use in treating a subject having a brain injury.
62) A composition comprising an agent that upmodulates IL-33 for the use in treating a subject having a brain injury.
63) A method to identify a subject at risk of a poor functional prognosis after a brain injury, the method comprising;
a. obtaining a biological sample from a subject who has had a brain injury; and
b. measuring a level of sST2 in the biological sample;
wherein the subject is considered at risk of a hemorrhagic transformation when the level of sST2 in the biological sample is increased as compared to the reference level.
64) The method of paragraph 63, wherein the level of sST2 is increased at least 2-fold, at least 3-fold, at least 4-fold, at least 5-fold, at least 6-fold, at least 7-fold, at least 8-fold, at least 9-fold, at least 10-fold, or more as compared to a reference level.
65) A method to identify a subject at risk of a poor functional prognosis after an ischemic stroke, the method comprising;
a. obtaining a biological sample from a subject who has had an ischemic stroke; and
b. measuring a level of sST2 in the biological sample;
wherein the subject is considered at risk of a poor functional prognosis when the level of sST2 is equal to or greater than 85 ng/mL.
66) A method to identify a subject at risk of a poor functional prognosis after a subarachnoid hemorrhage, the method comprising;
a. obtaining a biological sample from a subject who has had a subarachnoid hemorrhage; and
b. measuring a level of sST2 in the biological sample;
wherein the subject is considered at risk of poor functional prognosis when the level of sST2 is equal to or greater than 50 ng/mL.
67) A method to identify a subject at risk of a hemorrhagic transformation following a brain injury, the method comprising;
a. obtaining a biological sample from a patient who has had a brain injury; and
b. measuring a level of sST2 in the biological sample;
wherein the subject is considered at risk of a hemorrhagic transformation when the level of sST2 in the biological sample is increased as compared to the reference level.
68) The method of paragraph 65, wherein the level of sST2 is increased at least 2-fold, at least 3-fold, at least 4-fold, at least 5-fold, at least 6-fold, at least 7-fold, at least 8-fold, at least 9-fold, at least 10-fold, or more as compared to a reference level.
69) The method of paragraphs 63-68, further comprising administering to a subject at risk of a poor function prognosis the composition of paragraph 61 or paragraph 62.
70) The method of paragraphs 63-68, further comprising administering to a subject at risk of a poor function prognosis a prophylactic treatment.
71) A kit for detecting levels of sST2 after a brain injury, comprising:
a. an anti-sST2 antibody that binds to human sST2; and
b. instructions for detecting levels of sST2 in a biological sample.
72) The kit of paragraph 71, further comprising a device to collect a biological sample.
73) The kit of paragraph 71, further comprising a reference level.
74) A kit for detecting levels of sST2 after an ischemic stroke, comprising:
a. an anti-sST2 antibody that binds to human sST2; and
b. instructions for detecting levels of sST2 in a biological sample.
75) The kit of paragraph 74, further comprising a device to collect a biological sample.
76) The kit of paragraph 74, further comprising a reference level.
77) The kit of paragraph 74, wherein the instructions indicate that a person is at risk of a poor functional prognosis if the levels of sST2 in the biological sample are equal to or greater than 85 ng/mL.
78) A kit for detecting levels of sST2 after a subarachnoid hemorrhage, comprising:
a. an anti-sST2 antibody that binds to human sST2; and
b. instructions for detecting levels of sST2 in a biological sample.
79) The kit of paragraph 78, further comprising a device to collect a biological sample.
80) The kit of paragraph 78, further comprising a reference level.
81) The kit of paragraph 78, wherein the instructions indicate that a person is at risk of a poor functional prognosis if the levels of sST2 in the biological sample are equal to or greater than 50 ng/mL.

### EXAMPLES

### Example 1

Objective: ST2 is a member of the toll-like receptor superfamily that can alter inflammatory signaling of helper T-cells. It was investigated whether soluble ST2 (sST2) could independently predict outcome and hemorrhagic transformation (HT) in the setting of stroke.

Methods: sST2 was measured in patients enrolled in the Specialized Program of Translational Research in Acute Stroke (SPOTRIAS) network biomarker study. 646 patients had plasma samples collected at the time of hospital admission and 210 patients had a second sample collected 48 hours after stroke onset. Functional outcome was assessed using the modified Rankin Scale (mRS), with good and poor outcomes defined as mRS 0-2 and 3-6, respectively. HT was classified using ECASS criteria. The relationships between sST2, outcome, and HT were evaluated using multivariable logistic regression, Kaplan-Meier survival analysis and receiver operating characteristic curves.

Results: 646 patients were included in the analysis (mean age 69 years; 44% women), with a median NIHSS of 5 [IQR 2-12]. The median sST2 level on hospital admission was 35.0 ng/mL [IQR 25.7-49.8 ng/mL] and at 48 hours it was 37.4 ng/mL [IQR 27.9-55.6 ng/mL]. sST2 was independently associated with poor outcome (OR 2.77, 95% CI 1.54-5.06; p=0.003) and mortality (OR 3.56, 95% CI 1.58-8.38, p=0.001) after multivariable adjustment. Plasma sST2 was also associated with hemorrhagic transformation after adjustment for traditional risk factors (OR 5.58, 95% CI 1.40-37.44, p=0.039).

Interpretation: Soluble ST2 may serve as a prognostic biomarker for outcome and hemorrhagic transformation in patients with acute stroke. ST2 may link neuroinflammation and secondary injury after stroke.

### Example 2

Introduction

ST2 is a member of the Toll-like/Interleukin (IL)-1 receptor family.^{4,5} It serves as the receptor for IL-33 and integrates inflammation, tissue fibrosis and cardiac stress.⁵ A soluble form (sST2) is secreted into the circulation and functions as a decoy receptor for IL-33, inhibiting its signaling.⁶ Circulating levels of sST2 are associated with adverse outcome and mortality in patients with chronic heart failure (CHF) and myocardial infarction (MI).^{7,8,9} Recently, higher sST2 has been associated with an elevated risk of incident stroke.¹⁰

In murine models of ischemic stroke, the administration of interleukin-33 (IL-33) can ameliorate the pro-inflammatory response, reduce ischemic damage, and improve neurological function.¹¹ Accordingly, sST2 is highly expressed in astrocytes and microglia, two cells types that may participate in the post-ischemic inflammatory response.¹² However, no clinical study to date has evaluated the role of sST2 after ischemic stroke in patients. As a result, the relationship between circulating levels of sST2, functional outcome, and its potential for association with intraparenchymal brain pathology is unknown.

It was first sought to determine whether admission sST2 concentration independently predicted outcome and mortality in the setting of ischemic stroke. An association between sST2 and hemorrhagic transformation (HT) was also assessed.

Methods

Patient Characteristics. The subjects for the study were enrolled in the Specialized Programs of Translational Research in Acute Stroke (SPOTRIAS) network biomarker repository. The SPOTRIAS network repository enrolled patients age 18 years or greater who presented with symptoms consistent with ischemic stroke within 9 (median 3.62, IQR [2.02-4.87]) hours of last seen well between January 2007 and April 2010. Subjects were eligible if the NIH stroke scale (NIHSS) score was ≥1. Clinical data and plasma samples were provided by 6 academic stroke centers that participated in the SPOTRIAS biorepository. For the current analysis, patients who did not have baseline plasma samples (N=216) were excluded.

Modified Rankin Scale (mRS) assessments were prospectively gathered 90 days after the initial presentation through telephone interview with patients or family members. Poor outcome was defined as mRS of 3-6. Mortality was collected from the medical record and by accessing the Death Master File from the Social Security Administration.¹³ For cause of death analysis, the medical records of patients who died and were enrolled at the Partners Healthcare SPOTRIAS sites (Massachusetts General and Brigham and Women's Hospitals) were reviewed (N=314). The primary cause of death was classified based on consensus and categorized into neurological (including stroke, recurrent stroke, hemorrhage, or brain herniation), cardiac (sudden cardiac death), other, or unknown. All subjects or surrogates provided informed consent, and the study was approved by participating institutional review boards.

Soluble ST2 Analysis. Venous blood samples were collected in ethylenediaminetetraacetic acid (EDTA)-containing blood collection tubes. Within 1 hour, EDTA plasma was separated from cellular material via centrifugation, 2000 g for 15 minutes, and the supernatant was stored at −80° C. until the time of analysis. Soluble ST2 was measured from stored plasma samples using a commercially available enzyme-linked immunosorbent assay (Presage ST2 Assay Kit, Critical Diagnostics, San Diego, Calif.). The mean coefficient of variation for this assay is <5%. The lower and upper limits of detection of soluble ST2 were 3.1 ng/mL and 200.0 ng/mL, respectively.

Hemorrhagic Transformation Analysis. Imaging data was available for patients enrolled at the Partners Healthcare SPOTRIAS sites (Massachusetts General Hospital and Brigham & Women's Hospital; N=246). Hemorrhagic transformation was previously assessed¹⁴ using the European Cooperative Acute Stroke Study (ECASS) III criteria.¹⁵ All head CTs obtained through day 7 of the initial hospitalization were analyzed (median time to CT 1.2 days; IQR [1.0-2.1]), discrepancies were adjudicated by consensus, and those performing the imaging analysis were blinded to all clinical data. HT was dichotomized into the presence and absence of HT, and the presence or absence of hemorrhagic infarction type 2 (HI2) or greater. The latter classification was selected because hemorrhagic infarction type 2 (HI2), or parenchymal hemorrhage (PH1 and PH2) have been associated with worse long term functional outcome.¹⁶ Moreover, small petechial hemorrhagic infarction (HI1) may be benign and potentially serves as a marker of early reperfusion.17 Since symptomatic intracerebral hemorrhages (sICH), as defined by ECASS III criteria, were rare in this cohort (n=7), they were not separately studied.

Statistical Analysis. Baseline characteristics are expressed as mean±standard deviation (SD) for normally distributed continuous variables, or as median with interquartile range [IQR] for ordinal variables or continuous variables showing deviation from normality. Binary variables were represented as frequency and percentage. Skewed variables, such as NIHSS and glucose, were log-transformed to obtain normality prior to analysis. Odds ratios (OR) corresponded to a unit increase in the explanatory variable. Subjects were divided into tertiles based on sST2 concentration to quantify the effect size of the association between cohort characteristics and biomarker data. Differences between binary variables were analyzed using the Fisher's exact or chi-squared testing as appropriate. Continuous variables were compared between groups using analysis of variance (ANOVA) for parametric and Kruskal-Wallis for non-parametric testing.

Multivariable logistic regression was used to assess the independent association between plasma sST2 levels and functional outcome and mortality. To establish whether the associations between sST2 and functional outcome were independent of underlying cardiovascular disease, a series of multivariable models were designed to sequentially include risk factors for cardiovascular disease (i.e., atrial fibrillation, cardioembolic stroke subtype, and history of congestive heart failure). To avoid model overfitting for the logistic regression model predicting hemorrhagic transformation, only variables with a univariate p value<0.10 were included.

The discriminatory value of sST2 was analyzed using receiver operating characteristic (ROC) curve analysis. The incremental discriminatory value of sST2 was estimated by comparing the area under the ROC curve (AUC) for logistic regression models with and without sST2 included in the analysis. The ability of sST2 to reclassify risk was evaluated by comparing the prognostic accuracy of traditional clinical risk factors and calculating the net reclassification improvement (NRI) and integrated discrimination improvement (IDI) for models enriched with sST2. Continuous (category-free) NRI values in groups were calculated, since there were no pre-specified, externally validated risk categories.¹⁸⁻¹⁹ Kaplan-Meier survival curves were implemented to further investigate the ability of sST2 to predict mortality. Patients were stratified by sST2 tertile and cumulative event rates were compared with the log-rank test. All tests were two-sided and performed with the threshold for significance set at P<0.05. Statistical analysis was performed using JMP Pro 12.0 (SAS Institute, Cary, N.C., USA).

Results

Clinical Characteristics.

The initial study population consisted of 862 patients, however, 216 patients did not have baseline plasma samples available for analysis. A total of 646 patients comprised the primary study population. The mean age (±standard deviation) was 69±15 years, and 44% were female. The median admission NIHSS score was 5 [IQR 2-12], and the 90-day mRS score was 2 [IQR 1-4]. Plasma samples were collected at 7.1±3.3 hours after stroke onset, and the median sST2 concentration for the entire cohort was 35.0 ng/mL [IQR 25.7-49.8 ng/mL]. The median sST2 concentration for each tertile was 21.86 ng/mL [IQR 17.76-25.71 ng/mL], 34.98 ng/mL [IQR 31.62-39.40 ng/mL], and 60.24 ng/mL [IQR 49.71-76.89 ng/mL] respectively.

The clinical characteristics of the study population are presented in Table 1. Statistically significant differences between sST2 tertiles were observed for age, cardioembolic stroke subtype, history of atrial fibrillation, history of CHF, NIHSS score, and baseline glucose level. In healthy individuals, reported reference values for sST2 differ by sex.²⁰ Compared to the reported 95% upper reference limit for sST2 in females, the median sST2 for stroke cohort females was higher (38.9 ng/mL versus 33.5 ng/mL). In contrast, the median sST2 level in the stroke cohort males was lower than the reported 95% upper reference limit of sST2 for that sex (42.7 ng/mL versus 49.3 ng/mL). Consistent with these findings, 49% of females with stroke had elevated sST2 compared to 38% of males (p<0.001).

sST2 Predicts Outcome after Stroke.

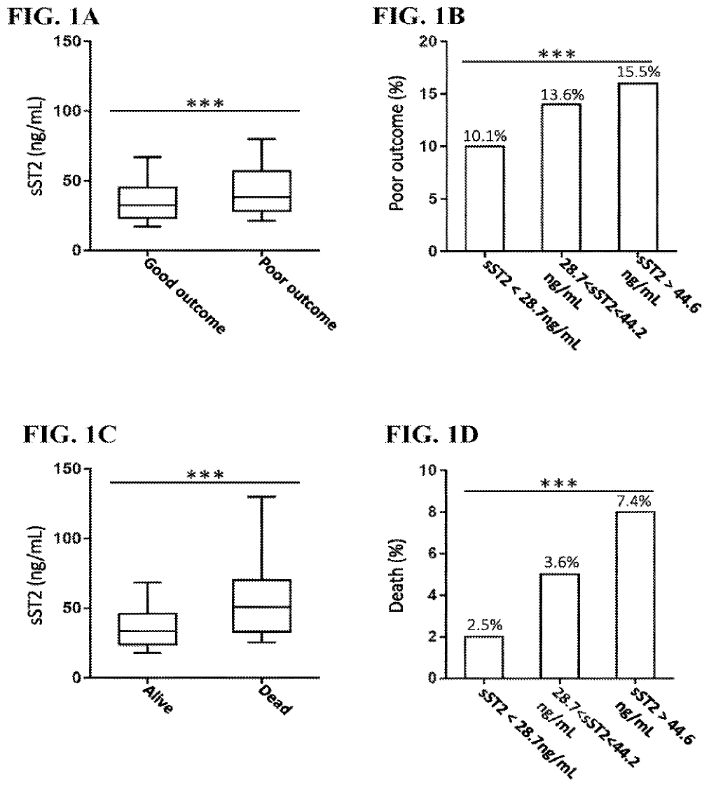

Univariate associations with poor outcome were assessed. Baseline NIHSS, glucose level, age, sex, history of atrial fibrillation, IV tPA use, history of CHF, and baseline sST2 tertile (OR 2.29; 95% CI 1.53-3.45; p=0.0003) were all associated with poor outcome (see Table 2). Univariate box plot distributions of sST2 level by outcome are shown in FIGS. 1-D. In multivariable logistic regression, baseline sST2 remained an independent predictor of poor outcome (OR 2.77; 95% CI 1.54-5.06; p=0.003; see Table 2). Additional significant predictors were age, baseline NIHSS, cardioembolic stroke subtype, IV tPA use, and glucose level.

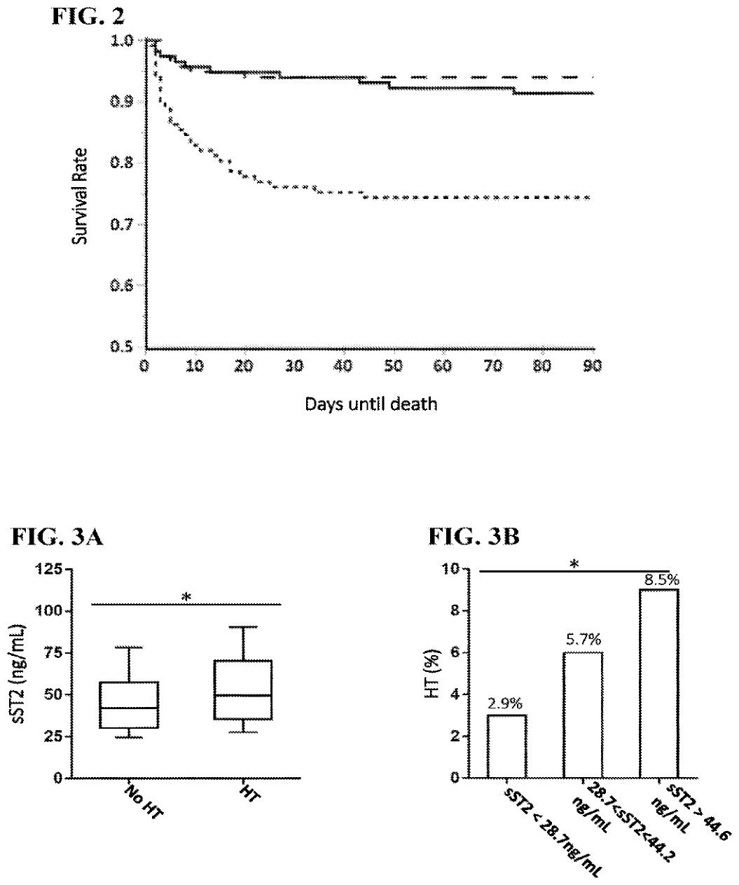

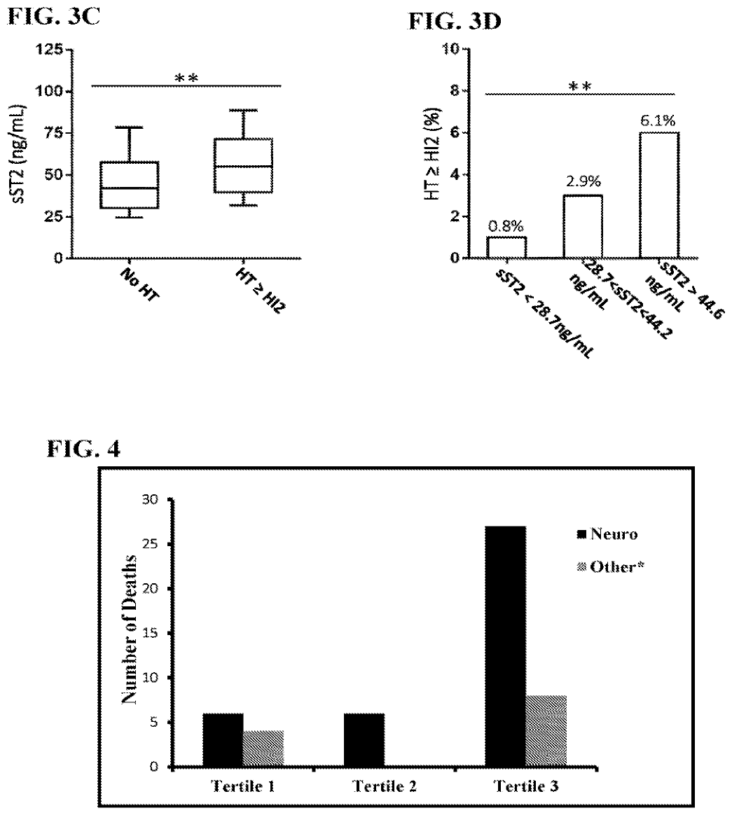

Predictors of mortality were next evaluated. Age, history of atrial fibrillation, history of congestive heart failure, IV tPA use, cardioembolic stroke subtype, baseline NIHSS, and baseline sST2 tertile (OR 3.86; 95% CI 2.15-7.26; p<0.0001) predicted 90-day mortality (Table 2). In multivariable analysis, baseline sST2 (OR 3.56; 95% CI 1.58-8.38; p=0.001) remained an independent predictor of death within 90 days after stroke (Table 2). Additional independent predictors included age and baseline NIHSS. The time to death was analyzed using Kaplan-Meier survival curves. Patients in the lowest and second tertiles of sST2 had a minimal risk of death. In contrast, patients with an sST2 level>44.6 ng/mL had the greatest risk of death (FIG. 2), which most often occurred within the first 30 days after the index stroke.

A subgroup of 314 patients had accessible medical records available for analysis to further investigate the cause of death. There were 51 deaths in this subgroup, out of a total of 86 in the entire cohort. Thirty-nine of 51 deaths were due to neurological causes (76%), 2 were due to cardiac causes (4%), 5 were attributed to non-neurological, non-cardiac causes (10%), and 5 were due to unknown causes (10%). The number of neurological deaths was significantly greater in the highest sST2 tertile compared with the lower sST2 tertiles (p=0.033; FIG. 4).

Evaluating the discriminatory capacity of sST2, ROC curves demonstrated better accuracy for the prediction of poor outcome and mortality when sST2 was added to major clinical risk factors.²¹ After addition of sST2 to baseline clinical characteristics, the AUC was 0.854 for poor outcome, and the AUC was 0.895 for mortality (see Table 5). Using net reclassification analysis, this was consistent with a small degree18,22 of reclassification for poor outcome (NRI=0.164) and a moderate degree of reclassification for mortality (NRI=0.478, see Table 5).

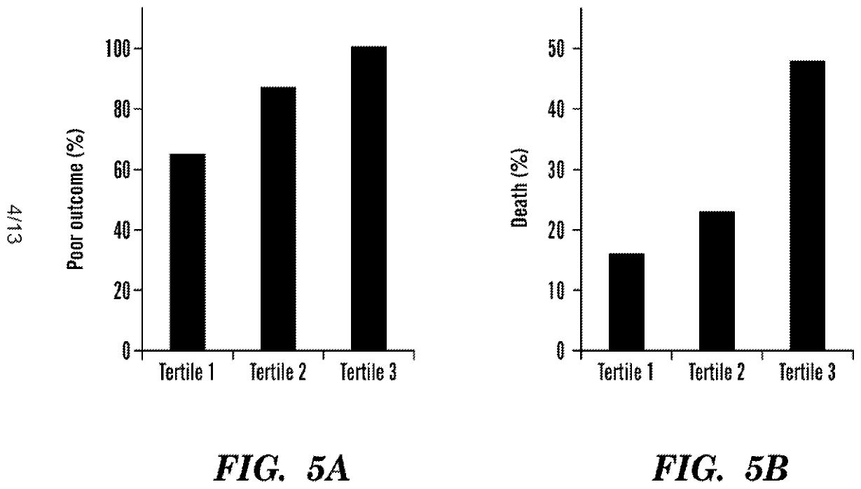

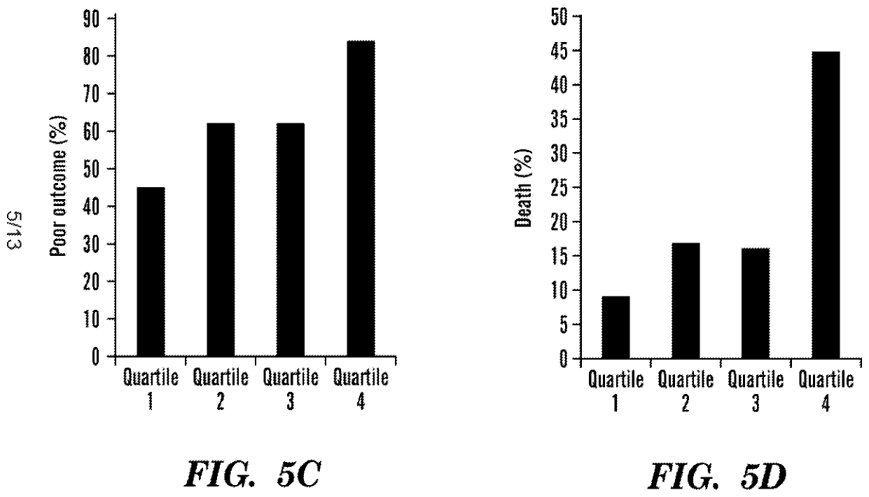

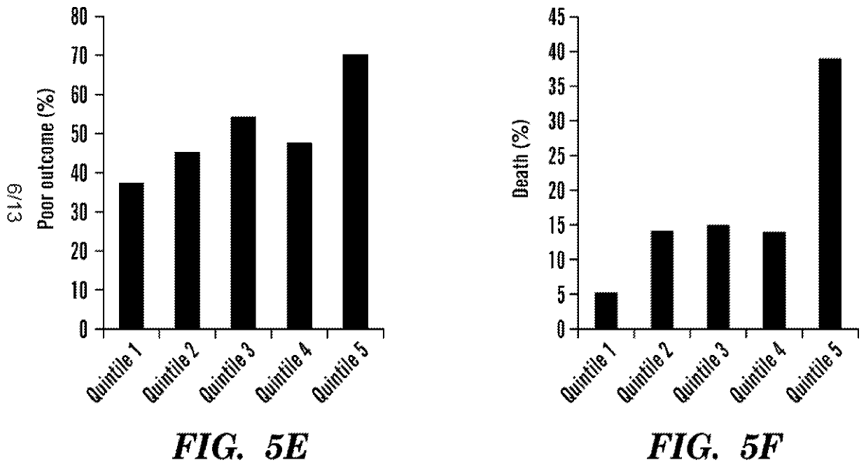

A series of sensitivity analyses were next performed. Analyses were repeated using sST2 quartiles and quintiles, and found that sST2 remained associated with outcome (all p<0.001; FIGS. 5A-5F). Lastly, in order to exclude a potential effect of differing sample storage time, sample storage time was included as a covariate in the multivariable model, and found that sST2 remained an independent predictor of outcome (Table 6).

sST2 Predicts Outcome and Mortality Independent from Cardiovascular Risk Factors.

Previous studies have identified sST2 as a prognostic biomarker in cardiovascular disease.^{7,8,9} In order to exclude the possibility that the association between sST2 and outcome was related to underlying cardiovascular disease, sequential multivariable models were developed that included cardiovascular disease risk factors. In all models developed, baseline sST2 remained an independent predictor of poor outcome (p<0.009; Table 3). Likewise, sST2 remained an independent predictor for 90-day mortality (p<0.002) in all models tested (Table 3). Admission values for ejection fraction and troponin were available in a subgroup of 314 patients. When these markers were included in the multivariable analysis, sST2 remained an independent predictor (p<0.049; Table 7).

sST2 Predicts Hemorrhagic Transformation after Stroke.

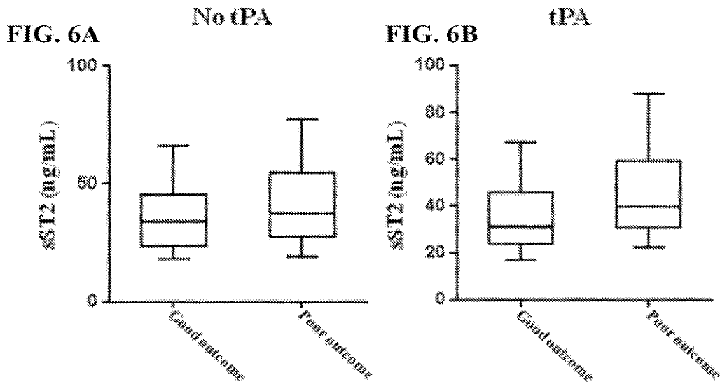

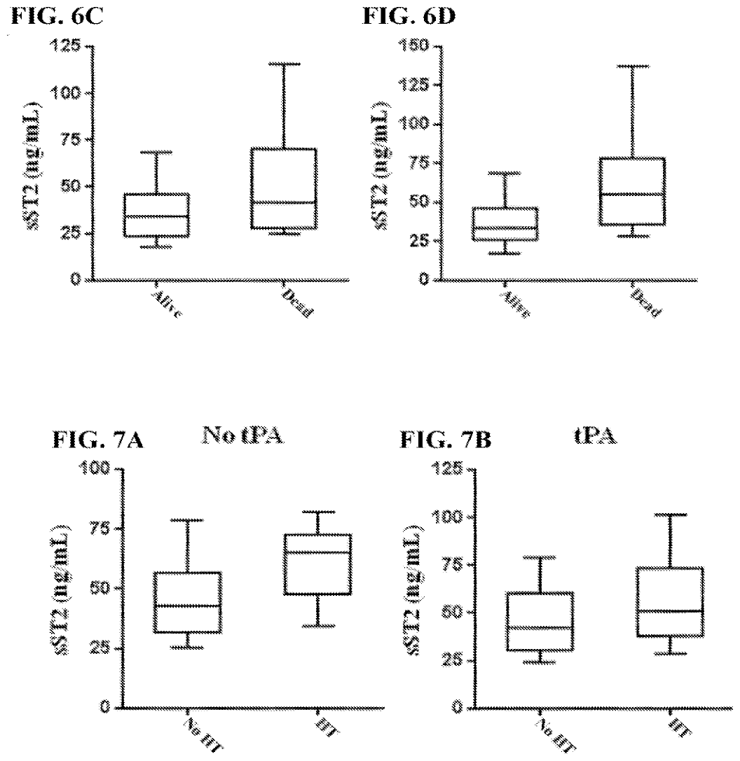

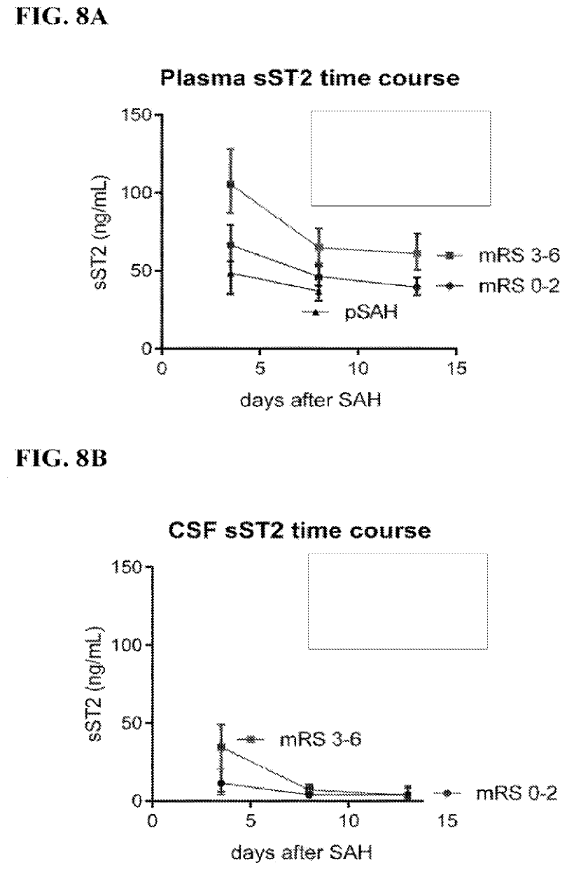

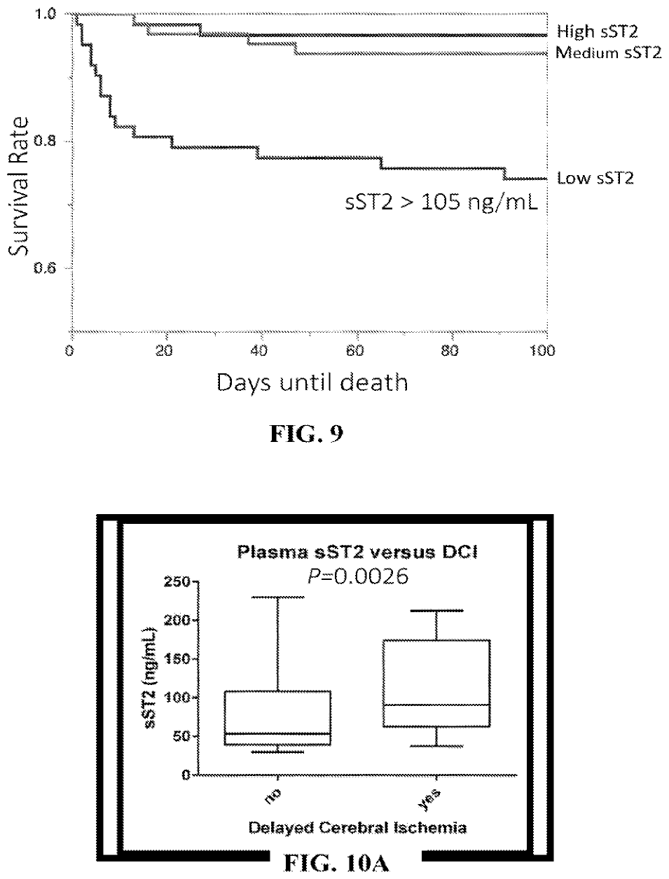

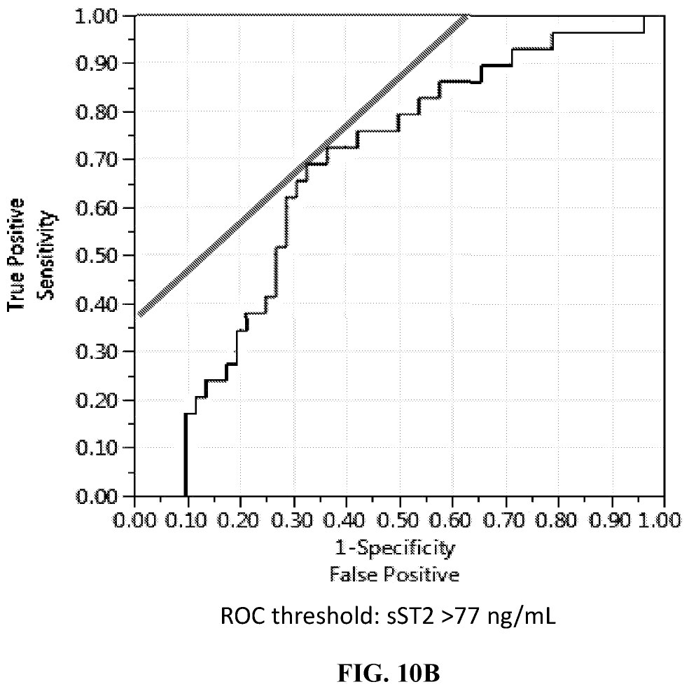

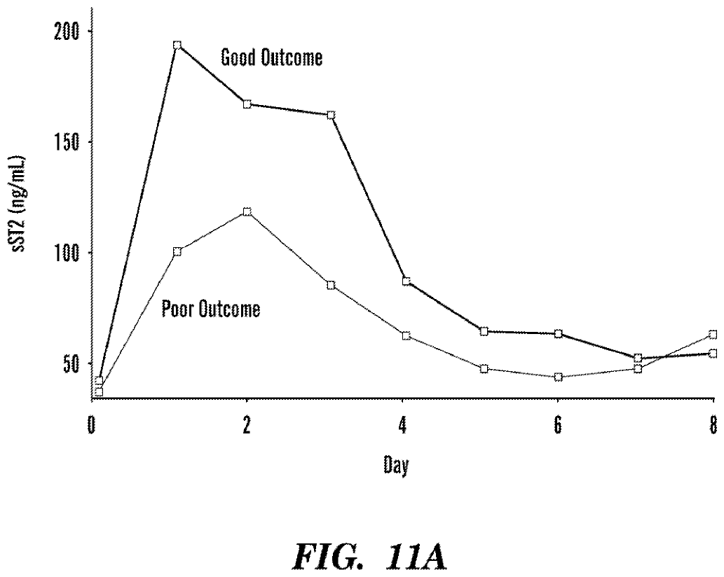

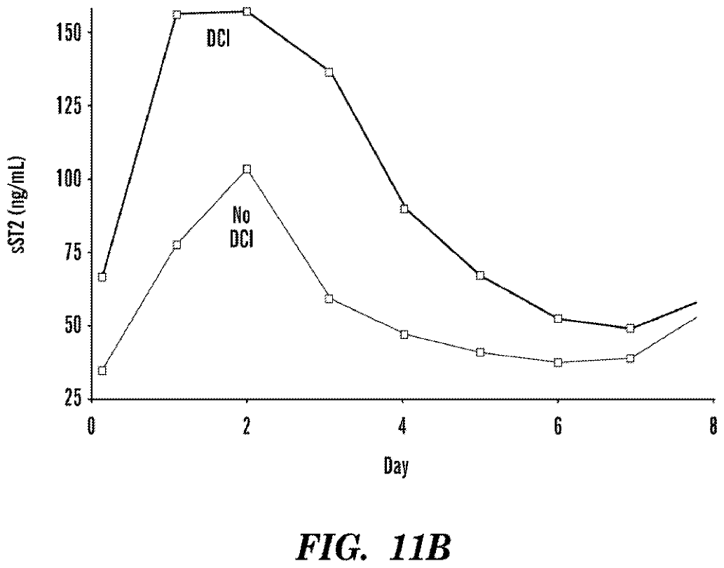

The relationship between sST2 and hemorrhagic transformation (HT) was analyzed. Of 246 patients with available scans, HT occurred in 42 (17%) patients; 23 (55%) with hemorrhagic infarction type 1 (HI1), 11 (26%) with hemorrhagic infarction type 2 (HI2), 4 (10%) with parenchymal hemorrhage type 1 (PH1) and 4 (10%) with parenchymal hemorrhage type 2 (PH2). Soluble ST2 level was significantly higher in patients with HT compared to those without (49.7 ng/mL vs. 42.1 ng/mL, p=0.03; FIG. 2). The HT rate by sST2 tertile was 3%, 6% and 9% respectively (p=0.049). In some studies, HI2 or greater has been proposed as a marker for post-stroke injury,¹⁶ whereas HI1 may be a beneficial marker of reperfusion.¹⁷ Accordingly, sST2 tertile predicted the risk of HI2 or greater (p=0.003). Table 4 shows the univariate associations between sST2 and HI2 or greater along with additional risks factors of HT including age, NIHSS, sex, baseline blood glucose, IV tPA, MMP-9 level, DWI volume, antiplatelet use, smoking history and anticoagulant use. Soluble ST2 remained an independent predictor of HI2 or greater (OR 5.40; 95% CI 1.40-35.61, p=0.039) when adjusted for NIHSS, baseline DWI volume, and anticoagulant use. When age, sex, baseline glucose level, IV tPA use, smoking history, antiplatelet use, or MMP-9 level were forced into the model, sST2 remained an independent predictor of HI2 or greater (p<0.050) in all models tested (Table 8). To further assess any potential impact of IV tPA on the ability of sST2 to predict HT, the cohort was divided into tPA-treated and non-tPA-treated subgroups. It was found that sST2 remained an independent predictor of HT in both groups (FIGS. 6A-D, 7A, and 7B). Finally, the inclusion of sST2 in models for predicting HI2 or greater had an AUC of 0.747 and was associated with a modest improvement in reclassification (NRI>0.268; Table 5).

sST2 Concentration at 48 Hours is Similar to Admission sST2.

210 patients of the original 646 had a second serial plasma sample collected 48 hrs after stroke onset available for analysis. The median 48 hour sST2 concentration for these subjects was 37.4 ng/mL [IQR 27.9-55.6 ng/mL], which was also associated with poor outcome (OR 5.84; 95% CI 2.98-12.52; p<0.0001) and mortality (OR 9.18; 95% CI 3.99-24.53; p<0.0001). sST2 measured at 48 hours remained an independent predictor of poor outcome and mortality, after adjusting for the same previously described clinical risk factors (Table 9). Lastly, the relationship between 48 h sST2 and hemorrhagic transformation was examined. Elevated sST2 levels 48 h after stroke onset was significantly associated with HT (OR 2.86; 95% CI 1.37-6.18; p=0.005) and HI2 or greater (OR 2.95; 95% CI 1.25-7.03; p=0.014).

Discussion

It was found that baseline plasma levels of sST2 independently predicted poor outcome, mortality and HT in patients who present with acute ischemic stroke. Importantly, sST2 was measured in blood samples obtained shortly after presentation to the emergency department, and predicts subsequent clinical events that can aid in risk stratification²³. Moreover, the association with outcome remained significant after adjusting for age, sex, NIHSS score, as well as a history of cardiovascular disease (e.g., atrial fibrillation and CHF). Furthermore, sST2 predicted the subsequent development of HT, which was independent of other factors known to be associated with HT (age, sex, NIHSS, admission glucose, anticoagulant use, smoking history, antiplatelet use, DWI volume, MMP-9, and IV tPA use). In the subset of subjects with serial samples available for analysis, sST2 level 48 hours after stroke onset was also associated with outcome, mortality, and HT.

Circulating sST2 was previously identified as a prognostic marker in heart failure and myocardial infarction.^{7,8,9,24} Together with other cardiac biomarkers such as troponin25 and B-type natriuretic peptide (BNP),^{26,27} data presented herein highlight overlapping injury responses that occur following both cerebrovascular and cardiovascular injury. There are several possible interpretations of findings presented herein in this context. Without wishing to be bound by a particular theory, sST2 may be a marker for stroke-induced cardiac injury, which in turn, influences subsequent neurological outcome after stroke. Alternatively, it is possible that sST2 may integrate both stroke severity and cardiovascular disease, each of which is strongly associated with outcome after stroke.²⁸⁻³⁰ Although data presented herein cannot exclude these possibilities, multivariable models described herein adjusting for cardiovascular risk factors and stroke severity indicate that sST2 is independent from these factors. Alternatively, it is possible that sST2 may reflect a separate and specific response to cerebral infarction, for example serving as a marker for neuroinflammation.

In this regard, it was also found that elevated sST2 circulating levels were associated with HT. Several preclinical studies and biomarker analyses have suggested that neuroinflammatory markers are linked to blood-brain barrier (BBB) breakdown and HT risk.^{11,31-33} Disruption of the BBB has been shown to be associated with neuroinflammation following ischemic injury,³² and hemorrhagic transformation has been reported as a maker of BBB breakdown.³³ The ligand for ST2, IL-33, is hypothesized to serve as an acute inflammatory signaling molecule that participates in maintaining barrier function and integrity.³⁴ Isoforms of ST2 and IL-33 are highly expressed in the brain and spinal cord, suggesting that IL-33/ST2 may function directly in the central nervous system.¹² In a murine model of stroke, IL-33 signaling through the membrane-bound form of ST2 has been shown to be neuroprotective through anti-inflammatory effects.³⁵ In contrast, the circulating soluble form (sST2), which was measured herein, is thought to antagonize IL-33 signaling⁶ and augment neuroinflammation by operating as a decoy receptor. Findings presented herein show that sST2 is associated with HT is consistent with this hypothesis.

```
Nucleic acid sequence encoding sST2
(SEQ ID NO: 1)
                                                            at gtattcaaca
gtatctggat cagaaaaaaa ttccaaaatt tattgtccta
ccattgacct ctacaactgg acagcacctc ttgagtggtt
taagaattgt caggctcttc aaggatcaag gtacagggcg
cacaagtcat ttttggtcat tgataatgtg atgactgagg
acgcaggtga ttacacctgt aaatttatac acaatgaaaa
tggagccaat tatagtgtga cggcgaccag gtccttcacg
gtcaaggatg agcaaggctt ttctctgttt ccagtaatcg
gagcccctgc acaaaatgaa ataaaggaag tggaaattgg
aaaaaacgca aacctaactt gctctgcttg ttttggaaaa
ggcactcagt tcttggctgc cgtcctgtgg cagcttaatg
gaacaaaaat tacagacttt ggtgaaccaa gaattcaaca
agaggaaggg caaaatcaaa gtttcagcaa tgggctggct
tgtctagaca tggttttaag aatagctgac gtgaaggaag
aggatttatt gctgcagtac gactgtctgg ccctgaattt
ctga (SEQ ID NO: 1)
Nucleic acid sequence encoding IL-33
(SEQ ID NO: 2)
                at gaagcctaaa atgaagtatt caaccaacaa
aatttccaca gcaaagtgga agaacacagc aagcaaagcc
ttgtgtttca agctgggaaa atcccaacag aaggccaaag
aagtttgccc catgtacttt atgaagctcc gctctggcct
tatgataaaa aaggaggcct gttactttag gagagaaacc
accaaaaggc cttcactgaa aacaggtaga aagcacaaaa
gacatctggt actcgctgcc tgtcaacagc agtctactgt
ggagtgcttt gcctttggta tatcaggggt ccagaaatat
actagagcac ttcatgattc aagtatcaca gataaggtgt
tactgagtta ctatgagtct caacacccct caaatgaatc
aggtgacggt gttgatggta agatgttaat ggtaaccctg
agtcctacaa aagacttctg gttgcatgcc aacaacaagg
aacactctgt ggagctccat aagtgtgaaa aaccactgcc
agaccaggcc ttctttgtcc ttcataatat gcactccaac
tgtgtttcat ttgaatgcaa gactgatcct ggagtgttta
taggtgtaaa ggataatcat cttgctctga ttaaagtaga
ctcttctgag aatttgtgta ctgaaaatat cttgtttaag
ctctctgaaa cttag (SEQ ID NO: 2)
```

Summary

Provided herein is evidence that elevated sST2 is associated with worse 90-day outcome, higher mortality, and increased risk of HT after acute ischemic stroke. These findings highlight the ST2 pathway as a candidate link to neuroinflammation-induced secondary injury.

### REFERENCES

1. Mozaffarian D, Benjamin E J, Go A S, et al. Heart Disease and Stroke Statistics-2015 Update: A Report From the American Heart Association. Circulation 2014; 131(4):e29-322.
2. Hankey G J. Long-term outcome after ischaemic stroke/transient ischaemic attack. Cerebrovasc. Dis. 2003; 16 Suppl 1(Suppl. 1):14-9.
3. Jickling G C, Sharp F R. Biomarker panels in ischemic stroke. Stroke. 2015; 46(3):915-20.
4. Schmitz J, Owyang A, Oldham E, et al. IL-33, an interleukin-1-like cytokine that signals via the IL-1 receptor-related protein ST2 and induces T helper type 2-associated cytokines. Immunity 2005; 23(5):479-90.
5. Kakkar R, Lee R T. The IL-33/ST2 pathway: therapeutic target and novel biomarker. Nat. Rev. Drug Discov. 2008; 7(10):827-40.
6. Hayakawa H, Hayakawa M, Kume A, Tominaga S. Soluble ST2 blocks interleukin-33 signaling in allergic airway inflammation. J. Biol. Chem. 2007; 282(36):26369-80.
7. Shimpo M, Morrow D A, Weinberg E O, et al. Serum Levels of the Interleukin-1 Receptor Family Member ST2 Predict Mortality and Clinical Outcome in Acute Myocardial Infarction. Circulation 2004; 109(18):2186-2190.
8. Ky B, French B, McCloskey K, et al. High-sensitivity ST2 for prediction of adverse outcomes in chronic heart failure. Circ. Hear. Fail. 2011; 4(2):180-187.
9. Sabatine M S, Morrow D A, Higgins U, et al. Complementary roles for biomarkers of biomechanical strain ST2 and N-terminal prohormone B-type natriuretic peptide in patients with ST-elevation myocardial infarction. Circulation 2008; 117(15):1936-1944.
10. Andersson C, Preis S R, Beiser A, et al. Associations of Circulating Growth Differentiation Factor-15 and ST2 Concentrations with Subclinical Vascular Brain Injury and Incident Stroke. Stroke 2015; 46(9):2568-2575.
11. Korhonen P, Kanninen K M, Lehtonen Š, et al. Immunomodulation by interleukin-33 is protective in stroke through modulation of inflammation. Brain Behav. Immun. 2016; 49(2015):322-336.
12. Yasuoka S, Kawanokuchi J, Parajuli B, et al. Production and functions of IL-33 in the central nervous system. Brain Res. 2011; 1385:8-17.
13. Nalichowski R, Keogh D, Chueh H C, Murphy S N. Calculating the benefits of a Research Patient Data Repository. AMIA Annu. Symp. Proc. 2006; 2006:1044.
14. Jha R, Battey T W K, Pham L, et al. Fluid-attenuated inversion recovery hyperintensity correlates with matrix metalloproteinase-9 level and hemorrhagic transformation in acute ischemic stroke. Stroke 2014; 45(4):1040-1045.
15. Hacke W, Kaste M, Bluhmki E, et al. Thrombolysis with alteplase 3 to 4.5 hours after acute ischemic stroke. N. Engl. J. Med. 2008; 359(13):1317-29.
16. Dzialowski I, Pexman J H W, Barber P A, et al. Asymptomatic hemorrhage after thrombolysis may not be benign: prognosis by hemorrhage type in the Canadian alteplase for stroke effectiveness study registry. Stroke. 2007; 38(1):75-9.
17. Molina C A, Alvarez-Sabin J, Montaner J, et al. Thrombolysis-Related Hemorrhagic Infarction: A Marker of Early Reperfusion, Reduced Infarct Size, and Improved Outcome in Patients With Proximal Middle Cerebral Artery Occlusion. Stroke 2002; 33(6):1551-1556.
18. Pencina M J, D'Agostino R B, Pencina K M, et al. Interpreting incremental value of markers added to risk prediction models. Am. J. Epidemiol. 2012; 176(6):473-81.
19. Pencina M J, D'Agostino R B, Steyerberg E W. Extensions of net reclassification improvement calculations to measure usefulness of new biomarkers. Stat. Med. 2011; 30(1):11-21.
20. Lu J, Snider J V, Grenache D G. Establishment of reference intervals for soluble ST2 from a United States population. Clin. Chim. Acta. 2010; 411(21-22):1825-6.
21. Feigin V L, Lawes C M M, Bennett D A, Anderson C S. Stroke epidemiology: a review of population-based studies of incidence, prevalence, and case-fatality in the late 20th century. Lancet. Neurol. 2003; 2(1):43-53.
22. Cook N R. Methods for evaluating novel biomarkers—a new paradigm. Int. J. Clin. Pract. 2010; 64(13):1723-7.
23. Mayeux R. Biomarkers: potential uses and limitations. NeuroRx 2004; 1(2):182-8.
24. Mueller T, Dieplinger B, Gegenhuber A, et al. Increased plasma concentrations of soluble ST2 are predictive for 1-year mortality in patients with acute destabilized heart failure. Clin. Chem. 2008; 54(4):752-6.
25. Ay H, Arsava E M, Sariba O. Creatine kinase-MB elevation after stroke is not cardiac in origin: comparison with troponin T levels. Stroke. 2002; 33(1):286-9.
26. Rost N S, Biffi A, Cloonan L, et al. Brain natriuretic peptide predicts functional outcome in ischemic stroke. Stroke. 2012; 43(2):441-5.
27. Chen X, Zhan X, Chen M, et al. The Prognostic Value of Combined NT-pro-BNP Levels and NIHSS Scores in Patients with Acute Ischemic Stroke. Intern. Med. 2012; 51(20):2887-2892.
28. Muir K W, Weir C J, Murray G D, et al. Comparison of neurological scales and scoring systems for acute stroke prognosis. Stroke. 1996; 27(10):1817-20.
29. Adams H P, Davis P H, Leira E C, et al. Baseline NIH Stroke Scale score strongly predicts outcome after stroke: A report of the Trial of Org 10172 in Acute Stroke Treatment (TOAST). Neurology 1999; 53(1): 126-31.
30. Appelros P, Nydevik I, Viitanen M. Poor outcome after first-ever stroke: predictors for death, dependency, and recurrent stroke within the first year. Stroke. 2003; 34(1):122-6.
31. Iadecola C, Anrather J. The immunology of stroke: from mechanisms to translation. Nat. Med. 2011; 17(7):796-808.
32. Yang Y, Rosenberg G A. Blood-brain barrier breakdown in acute and chronic cerebrovascular disease. Stroke 2011; 42(11):3323-3328.
33. Hamann G F, Okada Y, delZoppo G J, del Zoppo G J. Hemorrhagic transformation and microvascular integrity during focal cerebral ischemia/reperfusion. J. Cereb. blood flow Metab. 1996; 16(6):1373-8.
34. Martin N T, Martin M U. Interleukin 33 is a guardian of barriers and a local alarmin. Nat. Immunol. 2016; 17(2): 122-31.
35. Luo Y, Zhou Y, Xiao W, et al. Interleukin-33 ameliorates ischemic brain injury in experimental stroke through promoting Th2 response and suppressing Th17 response. Brain Res. 2015; 1597(13):86-94.
36. Dieplinger B, Egger M, Poelz W, et al. Long-term stability of soluble ST2 in frozen plasma samples. Clin. Biochem. 2010; 43(13-14):1169-70.
37. Ioannidis J P A, Panagiotou O A. Comparison of effect sizes associated with biomarkers reported in highly cited individual articles and in subsequent meta-analyses. JAMA 2011; 305(21):2200-10.

### Example 3

Introduction: Soluble ST2 (sST2) is a member of the Toll-like receptor superfamily implicated in pro-inflammatory signaling. It was investigated whether sST2 predicts delayed cerebral ischemia (DCI) and 90-day clinical outcome in patients with aneurysmal subarachnoid hemorrhage (SAH).

Methods: Soluble ST2 was measured in plasma samples from 182 patients who presented to a single institution with SAH. Blood samples were collected between days 1 and 5 after onset of SAH, and prior to the onset of DCI. Functional outcome was assessed at 3 months using the modified Rankin Scale (mRS) with good and poor outcome defined as mRS 0-2 and 3-6, respectively. Using a consensus definition, DCI was defined as a 2-point drop in GCS over a sustained period in patients whose clinical deterioration could not be explained by another cause. The relationships between sST2 level, DCI and outcome were assessed in univariable analysis. Multivariable logistic regression, Kaplan-Meier survival analysis, and receiver operating characteristic curves were used to determine the ability of sST2 to predict outcome and mortality. These findings were tested for replication in an independent cohort of 51 SAH patients recruited from a separate institution.

Results: The discovery cohort consisted of 182 subjects (mean age 56±12 years, 61% women). Elevated plasma sST2 predicted the development of DCI (OR 2.10, 95% 1.06-4.18 CI, P=0.0295), which remained an independent predictor after adjustment for age, World Federation of Neurological Surgeons (WFNS) grading score, modified Fisher score, intra-arterial vasodilator therapy and hydrocephalus (OR 2.78, 95% CI 1.00-7.97, p=0.0453). Elevated sST2 was also independently associated with poor 90-day functional outcome (OR 2.65, 95% CI 1.07-6.59, P=0.0304), and mortality (OR 5.36, 95% CI 1.48-19.39, p=0.0039) when adjusted for age, WFNS score, modified Fisher score and hydrocephalus. In the replication SAH cohort (N=51, 88% women), sST2 level was an independent predictor of DCI (OR 5.02, 95% CI 1.46-17.20, p=0.0034) and outcome at 90 days (OR 4.24, 95% CI 1.31-13.74, p=0.0072).

Conclusion: Plasma sST2 level predicted risk of DCI, 90-day outcome, and mortality after SAH.

### Example 4

Introduction

Aneurysmal subarachnoid hemorrhage is a devastating condition that affects a younger population and is associated with significant long-term disability¹. The high case fatality rate along with a lack of clinical consensus surrounding the management and pathophysiology of subarachnoid hemorrhage (SAH) underscores the need to establish biological markers that can identify patients at risk for developing delayed cerebral ischemia (DCI), aid in risk stratification, and inform treatment decisions².

The pathophysiology of subarachnoid hemorrhage is multifactorial, but pro-inflammatory alterations have been implicated in most sequelae of SAH including cerebral vasospasm, cortical spreading depression, and DCI.3-5 ST2 is a member of the Toll-like/Interleukin-1 receptor family that is expressed on macrophages and T helper (Th) cells⁶. The functional ligand for ST2, IL-33, was first identified when its expression was induced in canine endothelial cells in response to subarachnoid hemorrhage⁷. ST2/IL-33 binding stimulates Th cells to adopt an anti-inflammatory Th2 cellular identity⁸⁻⁹. Accordingly, IL-33 has been shown to reduce tissue injury and improve outcome in a murine model of stroke¹⁰. A soluble form (sST2) is secreted into the circulation and functions as a decoy receptor that sequesters IL-33 and blocks membrane bound signaling¹¹. This inhibition tips the immune response away from the Th-2 inflammatory response toward a Th1 pro-inflammatory response. Th1 activation has been associated with pro-inflammatory cytokines associated with Th1 activation have been associated with clinical severity at admission, DCI, and poor functional outcome in patients with subarachnoid hemorrhage^{2,5,12,13}. Likewise, elevated sST2 has been associated with increased microvascular hemorrhage dysfunction and poor functional outcome in ischemic stroke¹⁴. However, the relationship between circulating levels of sST2, functional outcome, and the intraparenchymal response to subarachnoid hemorrhage is unknown.

Given the destructive consequence of excess immune activation in subarachnoid hemorrhage and the role of ST2 in modulating pro versus anti-inflammatory T-cell responses it was hypothesized that sST2 may be a candidate marker of neuroinflammation induced secondary injury in subarachnoid hemorrhage. To test this hypothesis, the relationship between sST2 and functional outcome in patients with SAH was examined and potential associations between sST2 and DCI were explored.

Methods

Patient Characteristics. The primary study cohort consisted of 182 patients with SAH who were consecutively enrolled at Massachusetts General Hospital between May 2011 and November 2016. Patients under the age of 18 and patients with non-aneurysmal causes of SAH, including trauma, mycotic aneurysms, and arteriovenous malformations were excluded. Clinical and demographic data was obtained through patient or surrogate interview and verified with medical records. For the current analysis, patients that did not have plasma samples were excluded from the analysis.

Outcome data (mRS) was prospectively collected 90 days after SAH using modified Rankin Sale through telephone interview with patients and family members. For the current analysis, an mRS score>2 was considered poor outcome. Mortality data was collected through patient medical record and verified using the Death Master File from the Social Security Administration. Baseline SAH severity was classified using WFNS and the modified Fisher Grading Scale for SAH (mFisher). DCI was defined as a 2-point drop in GSC over a 24-hour period in patients whose clinical deterioration could not be attributed to another cause. Clinical & demographic data were collected throughout the subjects' hospitalization by two researchers blinded to sST2 data and discrepancies were adjudicated by consensus.

Soluble ST2 Analysis. Plasma samples from enrolled subjects were collected on post-SAH days 1-5 and prior to the onset of DCI. Follow-up blood samples for surviving patients were collected on post-SAH days 5-10 and 10-14. Plasma samples were collected in ethylenediminetetraacetic acid (EDTA)-coated blood collection tubes. EDTA plasma was separated from cellular material through centrifugation (2000 g for 15 minutes) within one hour of the sample draw and the plasma supernatant was stored at −80° C. until the time of analysis. Soluble ST2 was measured using a commercially available enzyme-linked immunosorbent assay (Presage ST2 Assay Kit, Critical Diagnostics, San Diego, Calif.). The mean coefficient of variation is <5% and the range of detection for this assay was 3.1-200.0 ng/mL.

Statistical Analysis. In this cohort, continuous variables with a normal distribution are expressed as mean±standard deviation (SD). Nonparametric variables, such as sST2 and transcranial doppler velocity (TCD), were log-transformed and reported as median with interquartile range [IQR]. Odds ratios (OR) correspond to a unit increase in the explanatory variable. Fisher's exact or chi-squared tests were used to quantify binary associations. Continuous variables were compared using analysis of variance (ANOVA) for parametric and Kruskal-Wallis for non-parametric. linear mixed models with random effects were used to assess the association of global sST2 over time (measured repeatedly during post-SAH days 0-14) and 90-day outcome data. A Kaplan-Meier survival curve was implemented to further investigate the association between sST2 and mortality. Subjects were divided into tertiles based on sST2 level and the cumulative event rates were compared using the log-rank test. Multivariable logistic regression was used to study the independent association between plasma sST2 and outcome, mortality, and DCI. To avoid overfitting the models, only variables with P<0.1 were considered relevant for regression analysis.

The ability of sST2 to effectively predict response variables was studied using receiver operating characteristic (ROC) curves, net reclassification improvement (NRI), and integrated discriminatory improvement (IDI). The discriminatory value of sST2 was evaluated by measuring comparing the prognostic accuracy of traditional clinical risk factors with the area under the ROC curve, NRI, and IDI for models enriched with sST2. Since there were no externally validated risk categories, continuous (category-free) NRI analysis was used to evaluate the ability of sST2 to reclassify risk.

Results

The primary study population consisted of 182 subjects with a mean age of 56 years (±SD 12). At the time of admission, 27% of subjects presented with a Hunt Hess score greater than 3 (N=50), 33% had a WFNS score of 4 or greater (N=58), and 76% had a modified fisher score greater than 2 [N=144]. The median 90-day mRS score was 2 [IQR 1-3] and 42% of subjects developed DCI. The total mortality for this cohort was 15% (N=25). Plasma samples were collected prior to the onset of DCI with a medium time from SAH onset to sample collection of 3.58 days [IQR 2.65-4.51]. The median concentration of plasma sST2 was 75.05 [43.96-133.60]. Serial samples were collected on PBD 5-10 and PBD 10-14 for patients that remained in the ICU to study the association of sST2 levels over time with SAH outcome. The median sST2 levels for these periods were 48.3 [IQR 24.97-78.15] and 46.68 [IQR 31.07-70.87], respectively.

sST2 Predicts Outcome after Subarachnoid Hemorrhage.

Associations of SAH risk factors and sST2 levels with poor outcome were studied. Age, admission Hunt-Hess, WFNS, modified Fisher score, hydrocephalus, and plasma sST2 (OR 3.03, 95% CI 1.59-5.79, p=0.0003) were all associated with 90-day poor outcome (Table 2). To avoid overfitting the multivariable model, only variables associated with poor outcome with p>0.1 were included. In multivariable logistic regression, the independent predictors of poor outcome were age, hydrocephalus and sST2 (OR 2.65; 95% CI 1.07-6.59; p=0.0304). Elevated sST2 over time (measured repeatedly during post SAH days 0-14) was significantly associated with poor functional outcome (Estimate 0.13; 95% CI 0.05-0.22; 95% CI; p=0.002) after correcting for repeated measures.

Associations between study variables and mortality were next evaluated. Significant predictors of 90-day mortality included age, Hunt-Hess, WFNS, modified fisher, hydrocephalus, and plasma sST2 (OR 4.65; 95% CI 1.93-11.19; p=0.0001). After multivariable adjustment, plasma sST2 remained an independent predictor of mortality (OR 5.36; 95% CI 1.48-19.39; p=0.0039). Global sST2 measured repeatedly over PBD 0-14 was significantly associated with mortality (Estimate 0.13; 95% CI 0.05-0.21; p=0.0017).

Kaplan-Meier survival curves was implemented to further study associations between sST2 and mortality. For this analysis, subjects were divided into tertiles based on sST2 concentration to quantify the association between biomarker data and time-to-death. The median sST2 concentration for each tertile was 35.23 ng/mL [IQR 32.59-43.96], 74.66 ng/mL [IQR 62.87-85.77], and 174.80 ng/mL [IQR 132.63-229.48] respectively. Patients in the highest sST2 tertile had a significantly greater risk of death compared with patients in the lower sST2-level tertiles (Log-Rank=0.0002). Mortality most often occurred in the first 30-days following SAH for patients with the highest sST2 tertile (data not shown).

The discriminatory value of sST2 was compared with that of traditional SAH risk factors using AUC comparison, NRI, and IDI. The addition of sST2 to the baseline predictive model significantly improved the AUC (0.7754 [95% CI 0.69-0.94] without sST2 vs. 0.8035 [95% CI 0.69-0.97] with sST2; p=0.0003). Net reclassification analysis further demonstrated the ability of sST2 to improve a model's predictive capacity. Adding sST2 to a model consisting of age and baseline WFNS improved the model's ability to reclassify response variables (NRI=0.4998 and a IDI of 0.6337).

sST2 Predicts Delayed Cerebral Ischemia after Subarachnoid Hemorrhage.

The relationship between sST2 and delayed cerebral ischemia (DCI) was assessed. Of the 182 patients with SAH, 59 met the inclusion criteria for DCI. Plasma sST2 levels were significantly higher in patients who developed DCI compared with those that did not (OR 2.81; 95% CI 1.36-5.83; p=0.0034). Additional significant predictors of DCI included WFNS, intra-arterial (IA) vasospasm treatment, and hydrocephalus. Clinical factors associated with DCI with a p<0.10 were included in the multivariable analysis. Plasma sST2 remained an independent predictor of DCI (OR 3.06; 95% CI 1.04-9.06) when adjusted for Hunt-Hess, WFNS, modified fisher, IA vasospasm treatment, and hydrocephalus. Adding sST2 to a baseline model improved the model's AUC, NRI, and IDI consistent with a high-degree of reclassification¹⁵.

sST2 predicts DCI, outcome, and mortality in an independent replication cohort.

A second cohort consisting of 51 patients with SAH comprised the replication cohort. The mean age was 61±11 years, and 89% were female. The median WFNS was 2.5 [IQR 1-4] and the median 90-day mRS was 1 [IQR 0-4]. Plasma samples were taken consecutively for the first five days post SAH and the median sST2 value for each patient was used to explore associations between outcome variables. The median sST2 value for this cohort was 70.9 [IQR 44.4-125.1]. Plasma sST2 was associated with DCI in this model (OR 3.01; 95% CI 1.39-6.55). The association between sST2 and DCI was independent of age, sex, and modified fisher score in a multivariable model (OR 3.01; 95% CI 1.27-7.11). Additionally, sST2 was a strong predictor of outcome (OR 4.24; 95% CI 2.02-9.06) and mortality (OR 31.94; IQR 6.95-146.70) in this cohort. In multivariable analysis, sST2 remained an independent predictor of these variables when adjusted for known risk factors.

Discussion

In work presented herein, it was found that plasma sST2 levels in the first 5 days post-SAH were significantly associated with DCI and 90-day functional outcome in two independent SAH cohorts. The relationship between sST2 and DCI was independent of traditional risk factors including baseline severity (WFNS), modified fisher score, hydrocephalus, and vasospasm treatment. What is more, sST2 was measured in blood samples obtained prior to the onset of DCI. This timing indicates that sST2 may be useful in predicting DCI and calculating risk stratification for patients with SAH. Furthermore, sST2 was an independent predictor of 90-day mRS and mortality and the association was independent of age, WFNS, modified fisher score, hydrocephalus, and vasospasm treatment. Finally, reproducibility of these results in an independent SAH cohort provides evidence for strength of the relationship between sST2 and DCI, outcome, and mortality.

The acute inflammatory response to subarachnoid hemorrhage is well complex and multifaceted.¹⁶⁻²⁰ However, neuroinflammation has been implicated as a key contributor to secondary injury after SAH. Activation of Toll-like receptors by erythrocyte breakdown initiates both innate and adaptive immune responses.^{19,21} ST2 is a member of the Toll-like/Interleukin (IL)-1 receptor family that is associated with cells known to mediate both aspects of immune sequela²². More specifically, ST2 and its functional ligand, IL-33, have been implicated in disease states in which the balance between Th1 and Th2 cell types is disrupted.^{11,14,23-26} In subarachnoid hemorrhage, pro-inflammatory Th1 infiltration has been associated with generalized cerebral edema²⁷ and vasospasm.²⁸

Several studies have targeted neuroinflammation as a target for therapeutic intervention^{29,30}. Studies show (IL)-1 receptor antagonism can safely and effectively lower inflammatory cytokines in the CSF of SAH patients³⁰ and therapies aimed at inhibiting inflammation have been associated with improved outcomes.²⁹ Given sST2's role in promoting a pro-inflammatory Th1 cellular environment and the destructive consequence of excessive inflammation on secondary SAH injury, sST2 is a maker for neuroinflammation in this population.

Summary

Provide herein is evidence that plasma sST2 in the first 5 days following SAH is associated with subsequent development of DCI, 90-day poor outcome, and mortality in two independent study cohorts. Taken together, these findings indicate that the sST2/IL-33 pathway may be important in the development of neuroinflammation-induced secondary injury after SAH.

### REFERENCES

1. Sebha F a, Hou J, Pluta R M, Zhang J H. The Importance of Early Brain Injury after Subarachnoid Hemorrhage. Prog Neurobiol. 2012; 97(1): 14-37. doi:10.1016/j.pneurobio.2012.02.003. The.
2. Savarraj J P J, Parsha K, Hergenroeder G W, et al. Systematic model of peripheral inflammation after subarachnoid hemorrhage. Neurology. 2017; 88(16):1535-1545. doi:10.1212/WNL.0000000000003842.
3. Ghaemi A, Alizadeh L, Babaei S, et al. Astrocyte-mediated inflammation in cortical spreading depression. Cephalalgia. April 2017:33310241770213. doi:10.1177/0333102417702132.
4. Osuka K, Suzuki Y, Tanazawa T, et al. Interleukin-6 and development of vasospasm after subarachnoid haemorrhage. Acta Neurochir (Wien). 1998; 140(9):943-951. http://www.ncbi.nlm.nih.gov/pubmed/9842432. Accessed Aug. 4, 2017.
5. Chamling B, Gross S, Stoffel-Wagner B, et al. Early Diagnosis of Delayed Cerebral Ischemia: Possible Relevance for Inflammatory Biomarkers in Routine Clinical Practice? World Neurosurg. 2017; 104:152-157. doi:10.1016/j.wneu.2017.05.021.
6. Kakkar R, Lee R T. The IL-33/ST2 pathway: therapeutic target and novel biomarker. Nat Rev Drug Discov. 2008; 7(10):827-840. doi:10.1038/nrd2660.
7. Onda H, Kasuya H, Takakura K, et al. Identification of genes differentially expressed in canine vasospastic cerebral arteries after subarachnoid hemorrhage. J Cereb Blood Flow Metab. 1999; 19(11):1279-1288. doi:10.1097/00004647-199911000-00013.
8. Schmitz J, Owyang A, Oldham E, et al. IL-33, an interleukin-1-like cytokine that signals via the IL-1 receptor-related protein ST2 and induces T helper type 2-associated cytokines. Immunity. 2005; 23(5):479-490. doi: 10.1016/j.immuni.2005.09.015.
9. Hayakawa H, Hayakawa M, Kume A, Tominaga S. Soluble ST2 blocks interleukin-33 signaling in allergic airway inflammation. J Biol Chem. 2007; 282(36):26369-26380. doi:10.1074/jbc.M704916200.
10. Korhonen P, Kanninen K M, Lehtonen S, et al. Immunomodulation by interleukin-33 is protective in stroke through modulation of inflammation. Brain Behav Immun. 2016; 49(2015):322-336. doi:10.1016/j.bbi.2015.06.013.
11. Kakkar R, Lee R T. The IL-33/ST2 pathway: therapeutic target and novel biomarker. Nat Rev Drug Discov. 2008; 7(10):827-840. doi:10.1038/nrd2660.
12. Chou S H-Y, Feske S K, Atherton J, et al. Early Elevation of Serum Tumor Necrosis Factor-α Is Associated With Poor Outcome in Subarachnoid Hemorrhage. J Investig Med. 2012; 60(7):1054-1058. doi:10.2310/JIM.0b013e3182686932.
13. Youssef S, St?ve O, Patarroyo J C, et al. The HMG-CoA reductase inhibitor, atorvastatin, promotes a Th2 bias and reverses paralysis in central nervous system autoimmune disease. Nature. 2002; 420(6911):78-84. doi:10.1038/nature01158.
14. Wolcott Z, Batra A, Bevers M B, et al. Soluble ST2 predicts outcome and hemorrhagic transformation after acute stroke. Ann Clin Transl Neurol. July 2017. doi:10.1002/acn3.435.
15. Cook N R. Methods for evaluating novel biomarkers—a new paradigm. Int J Clin Pract. 2010; 64(13):1723-1727. doi:10.1111/j.1742-1241.2010.02469.x.
16. Provencio J J. Inflammation in subarachnoid hemorrhage and delayed deterioration associated with vasospasm: a review. Acta Neurochir Suppl. 2013; 115:233-238. doi:10.1007/978-3-7091-1192-542.
17. McGirt M J, Mavropoulos J C, McGirt L Y, et al. Leukocytosis as an independent risk factor for cerebral vasospasm following aneurysmal subarachnoid hemorrhage. J Neurosurg. 2003; 98(6):1222-1226. doi:10.3171/jns.2003.98.6.1222.
18. Yoshimoto Y, Tanaka Y, Hoya K. Acute systemic inflammatory response syndrome in subarachnoid hemorrhage. Stroke. 2001; 32(9):1989-1993. http://www.ncbi.nlm.nih.gov/pubmed/11546886. Accessed Sep. 25, 2017.
19. Miller B A, Turan N, Chau M, Pradilla G. Inflammation, Vasospasm, and Brain Injury after Subarachnoid Hemorrhage. Biomed Res Int. 2014; 2014:1-16. doi:10.1155/2014/384342.
20. Lucke-Wold B P, Logsdon A F, Manoranjan B, et al. Aneurysmal Subarachnoid Hemorrhage and Neuroinflammation: A Comprehensive Review. Int J Mol Sci. 2016; 17(4):497. doi:10.3390/ijms17040497.
21. Ascenzi P, Bocedi A, Visca P, et al. Hemoglobin and heme scavenging. IUBMB Life (International Union Biochem Mol Biol Life). 2005; 57(11):749-759. doi: 10.1080/15216540500380871.
22. Brint E K, Xu D, Liu H, et al. ST2 is an inhibitor of interleukin 1 receptor and Toll-like receptor 4 signaling and maintains endotoxin tolerance. Nat Immunol. 2004; 5(4):373-379. doi:10.1038/ni1050.
23. Ali M, Zhang G, Thomas W R, et al. Investigations into the role of ST2 in acute asthma in children. Tissue Antigens. 2009; 73(3):206-212. doi:10.1111/j.1399-0039.2008.01185.x.
24. Xu D, Jiang H-R, Kewin P, et al. IL-33 exacerbates antigen-induced arthritis by activating mast cells. Proc Natl Acad Sci USA. 2008; 105(31):10913-10918. doi:10.1073/pnas.0801898105.
25. Rudolf J W, Lewandrowski E L, Lewandrowski K B, Januzzi J L, Bajwa E K, Baron J M. ST2 Predicts Mortality and Length of Stay in a Critically Ill Noncardiac Intensive Care Unit Population. Am J Clin Pathol. 2016; 145(2):203-210. doi:10.1093/ajcp/aqv082.
26. Shimpo M, Morrow D A, Weinberg E O, et al. Serum Levels of the Interleukin-1 Receptor Family Member ST2 Predict Mortality and Clinical Outcome in Acute Myocardial Infarction. Circulation. 2004; 109(18):2186-2190. doi:10.1161/01.CIR.0000127958.21003.5A.
27. Sozen T, Tsuchiyama R, Hasegawa Y, et al. Immunological Response in Early Brain Injury After SAH. In: Early Brain Injury or Cerebral Vasospasm. Vol 110. Vienna: Springer Vienna; 2011:57-61. doi:10.1007/978-3-7091-0353-110.
28. Dietrich H H, Dacey R G. Molecular keys to the problems of cerebral vasospasm. Neurosurgery. 2000; 46(3):517-530. http://www.ncbi.nlm.nih.gov/pubmed/10719847. Accessed Sep. 25, 2017.
29. Chaichana K L, Pradilla G, Huang J, Tamargo R J. Role of inflammation (leukocyte-endothelial cell interactions) in vasospasm after subarachnoid hemorrhage. World Neurosurg. 2010; 73(1):22-41. doi:10.1016/j.surneu.2009.05.027.
30. Singh N, Hopkins S J, Hulme S, et al. The effect of intravenous interleukin-1 receptor antagonist on inflammatory mediators in cerebrospinal fluid after subarachnoid haemorrhage: a phase II randomised controlled trial. J Neuroinflammation. 2014; 11(1):1. doi:10.1186/1742-2094-11-1.

TABLE 1
Cohort characteristics according to sST2 tertile (n = 646).

| | | | sST2 Tertile | | | | | | |
|---|---|---|---|---|---|---|---|---|---|
| | | | First Tertile | | Second Tertile | | Third Tertile | | |
| | Entire Cohort | | sST2 ≤ 28.7_{ng/mL} | | 28.7 < sST2 ≤ 44.2_{ng/mL} | | sST2 > 44.6_{ng/mL} | | |
| Characteristic | (n = 646) | | (n = 215) | | (n = 216) | | (n = 215) | | P value |
| Age (Y), mean (SD) | 69 | (15) | 67.01 | (15) | 68.70 | (15.26) | 71.13 | (14.47) | 0.029 |
| Female, n (%) | 282 | (44%) | 97 | (45%) | 103 | (47%) | 82 | (38%) | 0.136 |
| Race, n (%) | | | | | | | | | |
| White | 578 | (89%) | 186 | (87%) | 196 | (91%) | 196 | (91%) | 0.222 |
| Black | 50 | (8%) | 23 | (11%) | 15 | (7%) | 12 | (6%) | 0.121 |
| Asian | 15 | (2%) | 6 | (3%) | 4 | (2%) | 5 | (2%) | 0.722 |
| Other | 3 | (1%) | 0 | (0%) | 1 | (0%) | 2 | (1%) | 0.337 |
| Cardioembolic stroke | 210 | (33%) | 46 | (21%) | 65 | (30%) | 99 | (46%) | <0.0001 |
| subtype | | | | | | | | | |
| Past medical history | | | | | | | | | |
| Hypertension | 464 | (72%) | 143 | (67%) | 163 | (75%) | 158 | (73%) | 0.085 |
| Diabetes | 141 | (22%) | 37 | (17%) | 48 | (22%) | 56 | (26%) | 0.093 |
| Hyperlipidemia | 55 | (14%) | 14 | (14%) | 20 | (14%) | 21 | (12%) | 0.749 |
| Afib | 173 | (27%) | 34 | (16%) | 50 | (23%) | 89 | (41%) | <0.0001 |
| CHF | 69 | (11%) | 12 | (6%) | 24 | (11%) | 33 | (16%) | 0.004 |
| NIHSS, median [IQR] | 5 | [2, 12] | 5 | [2, 10] | 5 | [2, 13] | 7 | [3, 14] | 0.009 |
| IV tPA, n (%) | 277 | (43%) | 79 | (37%) | 94 | (44%) | 104 | (49%) | 0.040 |
| Glucose, median [IQR] | 119 | [102, 140] | 113 | [100, 134] | 118 | [101, 140] | 124 | [106, 196] | 0.002 |

sST2 indicates soluble ST2; SD, standard deviation; Afib, Atrial Fibrillation; CHF, Congestive Heart Failure; NIHSS, National Institutes of Health Stroke Scale; IQR, interquartile range; IV tPA, intravenous tissue plasminogen activator.

TABLE 2
sST2 predicts poor outcome and mortality after ischemic stroke.

| Univariate Analysis | | | | Multivariate Analysis | | | |
|---|---|---|---|---|---|---|---|
| | Confidence Interval | | | | Confidence Interval | | |
| | Lower | Upper | | | Lower | Upper | |
| OR | 95% | 95% | P Value | OR | 95% | 95% | P Value |

Univariate and multivariate predictors of poor outcome after stroke (mRS 3-6).

| | | | | | | | | |
|---|---|---|---|---|---|---|---|---|
| Age (increase per year) | 1.04 | 1.02 | 1.05 | <0.0001 | 1.04 | 1.02 | 1.06 | <0.0001 |
| Sex (F) | 1.50 | 1.08 | 2.08 | 0.015 | 1.41 | 0.89 | 2.24 | 0.139 |
| Glucose (increase | 2.08 | 1.27 | 3.49 | 0.003 | 2.18 | 1.19 | 3.87 | 0.012 |
| per log unit) | | | | | | | | |
| CE | 1.15 | 0.81 | 1.63 | 0.425 | 0.52 | 0.31 | 0.87 | 0.014 |
| Afib | 2.14 | 1.49 | 3.10 | <0.0001 | 1.31 | 0.74 | 2.31 | 0.032 |
| CHF | 2.07 | 1.24 | 3.50 | 0.006 | 0.70 | 0.35 | 1.40 | 0.044 |
| IV tPA | 2.26 | 1.62 | 3.16 | <0.0001 | 0.56 | 0.34 | 0.90 | 0.017 |
| NIHSS (increase | 4.70 | 3.67 | 6.13 | <0.0001 | 5.79 | 4.25 | 8.09 | <0.0001 |
| per log unit) | | | | | | | | |
| sST2 (increase | 2.29 | 1.53 | 3.45 | 0.0003 | 2.77 | 1.54 | 5.06 | 0.003 |
| per tertile) | | | | | | | | |

Univariate and multivariate predictors of death after stroke.

| | | | | | | | | |
|---|---|---|---|---|---|---|---|---|
| Age (increase per year) | 1.08 | 1.05 | 1.10 | <0.0001 | 1.07 | 1.04 | 1.10 | <0.0001 |
| Sex (F) | 1.31 | 0.83 | 2.07 | 0.241 | 1.02 | 0.54 | 1.90 | 0.963 |
| Glucose (increase | 2.17 | 1.15 | 4.03 | 0.017 | 1.93 | 0.95 | 4.31 | 0.068 |
| per log unit) | | | | | | | | |
| CE | 1.74 | 1.09 | 2.76 | 0.021 | 0.66 | 0.33 | 1.28 | 0.220 |
| Afib | 3.36 | 2.10 | 5.41 | <0.0001 | 1.27 | 0.63 | 2.53 | 0.772 |
| CHF | 2.31 | 1.23 | 4.17 | 0.010 | 0.85 | 0.37 | 1.87 | 0.672 |
| IV tPA | 2.35 | 1.48 | 3.78 | 0.0003 | 0.79 | 0.42 | 1.49 | 0.469 |
| NIHSS (increase | 1.19 | 1.15 | 1.24 | <0.0001 | 7.23 | 4.53 | 12.35 | <0.0001 |
| per log unit) | | | | | | | | |
| sST2 (increase | 3.86 | 2.15 | 7.26 | <0.0001 | 3.56 | 1.58 | 8.38 | 0.001 |
| per tertile) | | | | | | | | |

Afib indicates Atrial Fibrillation; CHF, Congestive Heart Failure; NIHSS, National Institutes of Health Stroke Scale; CE, cardioembolic stroke subtype; IV tPA, intravenous tissue plasminogen activator; sST2, soluble ST2; OR, odds ratio.

TABLE 3
sST2 predicts outcome and mortality independent from cardiovascular risk factors.

| Multivariate models for predictors of poor outcome after stroke. | | | | | Multivariate models for predictors of death after stroke. | | | | |
|---|---|---|---|---|---|---|---|---|---|
| | | Confidence Interval | | | | | Confidence Interval | | |
| | | Lower | Upper | | | | Lower | Upper | |
| | OR | 95% | 95% | P Value | | OR | 95% | 95% | P Value |
| Model 1 | 2.29 | 1.53 | 3.45 | 0.001 | Model 1 | 3.83 | 2.13 | 7.21 | <0.0001 |
| Model 2 | 2.32 | 1.35 | 4.04 | 0.009 | Model 2 | 2.98 | 1.41 | 6.56 | 0.002 |
| Model 3 | 2.76 | 1.53 | 5.05 | 0.003 | Model 3 | 3.51 | 1.52 | 8.56 | 0.002 |
| Model 4 | 2.88 | 1.59 | 5.30 | 0.002 | Model 4 | 3.68 | 1.55 | 9.24 | 0.003 |
| Model 5 | 2.97 | 1.62 | 5.52 | 0.002 | Model 5 | 3.69 | 1.54 | 9.32 | 0.003 |

| | |
|---|---|
| Model 1: Unadjusted | Model 1: Unadjusted |
| Model 2: Adjusted for age, sex, NIHSS, glucose, | Model 2: Adjusted for age, sex, NIHSS, glucose, |
| IV tPA, site | IV tPA, site |
| Model 3: Adjusted for age, sex, NIHSS, glucose, | Model 3: Adjusted for age, sex, NIHSS, glucose, |
| IV tPA, site, Afib | IV tPA, site, Afib |
| Model 4: Adjusted for age, sex, NIHSS, glucose, | Model 4: Adjusted for age, sex, NIHSS, glucose, |
| IV tPA, site, Afib, CHF | IV tPA, site, Afib, CHF |
| Model 5: Adjusted for age, sex, NIHSS, glucose, | Model 5: Adjusted for age sex, NIHSS, glucose, |
| IV tPA, site, Afib, CHF, CE | IV tPA, site, Afib, CHF, CE |

NIHSS indicates National Institutes of Health Stroke Scale; IV tPA, intravenous tissue plasminogen activator; site, SPOTRIAS stroke site; Afib, Atrial Fibrillation; CHF, Congestive Heart Failure; CE, cardioembolic stroke subtype.

TABLE 4
sST2 predicts hemorrhagic transformation after ischemic stroke.

| | Univariate Analysis | | | | Multivariate Analysis | | | |
|---|---|---|---|---|---|---|---|---|
| | | Confidence | | | | Confidence | | |
| | | Interval | | | | Interval | | |
| | | Lower | Upper | | | Lower | Upper | |
| | OR | 95% | 95% | P Value | OR | 95% | 95% | P value |
| Age | 1.02 | 0.99 | 1.05 | 0.174 | | | | |
| NIHSS | 2.33 | 1.34 | 4.40 | 0.002 | 2.46 | 1.29 | 5.11 | 0.005 |
| Sex (F) | 0.47 | 0.17 | 1.17 | 0.108 | | | | |
| Glucose | 1.97 | 0.54 | 6.21 | 0.288 | | | | |
| IV tPA | 1.02 | 0.42 | 2.62 | 0.971 | | | | |
| MMP-9 | 0.95 | 0.61 | 1.54 | 0.827 | | | | |
| BL DWI | 1.30 | 1.00 | 1.74 | 0.052 | 1.00 | 0.99 | 1.01 | 0.738 |
| Antiplatelet use | 1.11 | 0.46 | 2.59 | 0.814 | | | | |
| Smoking history | 1.24 | 0.52 | 3.18 | 0.632 | | | | |
| Anticoagulant use | 3.40 | 1.21 | 8.78 | 0.022 | 3.80 | 1.22 | 11.41 | 0.022 |
| sST2 | 7.90 | 2.15 | 51.02 | 0.003 | 5.40 | 1.40 | 35.61 | 0.039 |

NIHSS indicates National Institutes of Health Stroke Scale; sST2, soluble ST2; IV tPA, intravenous tissue plasminogen activator; MMP-9, matrix metalloproteinase-9; BL DWI, baseline DWI volume.

TABLE 5
Discrimination analysis of sST2 for predicting poor
outcome, mortality, and hemorrhagic transformation.

| | | | | |
|---|---|---|---|---|
| Model | AUC | 95% CI | NRI | IDI |

Discrimination for poor outcome (mRS 3-6).

| | | | | |
|---|---|---|---|---|
| sST2 | 0.603 | 0.557-0.648 | — | — |
| Sex | 0.55 | 0.509-0.589 | Reference | Reference |
| Sex + sST2 | 0.619 | 0.573-0.664 | 0.263 | 0.0132 |
| Age | 0.667 | 0.623-0.710 | Reference | Reference |
| Age + sST2 | 0.678 | 0.634-0.720 | 0.251 | 0.0095 |
| NIHSS | 0.832 | 0.795-0.862 | Reference | Reference |
| NIHSS + sST2 | 0.837 | 0.800-0.867 | 0.158 | 0.0115 |
| Sex, Age, NIHSS | 0.851 | 0.817-0.880 | Reference | Reference |
| Sex, Age, NIHSS + | 0.854 | 0.820-0.883 | 0.164 | 0.0088 |
| sST2 | | | | |

Discrimination for mortality.

| | | | | |
|---|---|---|---|---|
| sST2 | 0.691 | 0.627-0.748 | — | — |
| Sex | 0.534 | 0.477-0.591 | Reference | Reference |
| Sex + sST2 | 0.697 | 0.632-0.755 | 0.570 | 0.002 |
| Age | 0.744 | 0.684-0.796 | Reference | Reference |
| Age + sST2 | 0.793 | 0.737-0.840 | 0.533 | 0.0005 |
| NIHSS | 0.852 | 0.806-0.889 | Reference | Reference |
| NIHSS + sST2 | 0.871 | 0.824-0.907 | 0.351 | 0.0014 |
| Sex, Age, NIHSS | 0.883 | 0.841-0.914 | Reference | Reference |
| Sex, Age, NIHSS + | 0.895 | 0.854-0.925 | 0.478 | 0.0014 |
| sST2 | | | | |

Discrimination for hemorrhagic transformation.

| | | | | |
|---|---|---|---|---|
| sST2 | 0.606 | 0.513-0.692 | — | — |
| Age | 0.606 | 0.498-0.704 | Reference | Reference |
| Age + sST2 | 0.668 | 0.561-0.759 | 0.577 | 0.0059 |
| NIHSS | 0.715 | 0.596-0.810 | Reference | Reference |
| NIHSS + sST2 | 0.741 | 0.628-0.828 | 0.268 | 0.0007 |
| Age & NIHSS | 0.726 | 0.610-0.818 | Reference | Reference |
| Age & NIHSS + sST2 | 0.747 | 0.638-0.832 | 0.268 | 0.0006 |

Discrimination for poor outcome (mRS 3-6).

| | | | | |
|---|---|---|---|---|
| sST2 | 0.603 | 0.557-0.648 | — | — |
| Sex | 0.55 | 0.509-0.589 | Reference | Reference |
| Sex + sST2 | 0.619 | 0.573-0.664 | 0.263 | 0.0132 |
| Age | 0.667 | 0.623-0.710 | Reference | Reference |
| Age + sST2 | 0.678 | 0.634-0.720 | 0.251 | 0.0095 |
| NIHSS | 0.832 | 0.795-0.862 | Reference | Reference |
| NIHSS + sST2 | 0.837 | 0.800-0.867 | 0.158 | 0.0115 |
| Sex, Age, NIHSS | 0.851 | 0.817-0.880 | Reference | Reference |
| Sex, Age, NIHSS + | 0.854 | 0.820-0.883 | 0.164 | 0.0088 |
| sST2 | | | | |

Discrimination for mortality.

| | | | | |
|---|---|---|---|---|
| sST2 | 0.691 | 0.627-0.748 | — | — |
| Sex | 0.534 | 0.477-0.591 | Reference | Reference |
| Sex + sST2 | 0.697 | 0.632-0.755 | 0.570 | 0.002 |
| Age | 0.744 | 0.684-0.796 | Reference | Reference |
| Age + sST2 | 0.793 | 0.737-0.840 | 0.533 | 0.0005 |
| NIHSS | 0.852 | 0.806-0.889 | Reference | Reference |
| NIHSS + sST2 | 0.871 | 0.824-0.907 | 0.351 | 0.0014 |
| Sex, Age, NIHSS | 0.883 | 0.841-0.914 | Reference | Reference |
| Sex, Age, NIHSS + | 0.895 | 0.854-0.925 | 0.478 | 0.0014 |
| sST2 | | | | |

Discrimination for hemorrhagic transformation.

| | | | | |
|---|---|---|---|---|
| sST2 | 0.606 | 0.513-0.692 | — | — |
| Age | 0.606 | 0.498-0.704 | Reference | Reference |
| Age + sST2 | 0.668 | 0.561-0.759 | 0.577 | 0.0059 |
| NIHSS | 0.715 | 0.596-0.810 | Reference | Reference |
| NIHSS + sST2 | 0.741 | 0.628-0.828 | 0.268 | 0.0007 |
| Age & NIHSS | 0.726 | 0.610-0.818 | Reference | Reference |
| Age & NIHSS + sST2 | 0.747 | 0.638-0.832 | 0.268 | 0.0006 |

AUC indicates area under the receiver operating characteristic curve; NRI, net reclassification improvement; IDI, integrated discrimination improvement; NIHSS, National Institutes of Health Stroke Scale; sST2, soluble ST2.

TABLE 6
sST2 independently predicts outcome and mortality when adjusted for sample storage time.

| | Outcome | | | | Mortality | | | |
|---|---|---|---|---|---|---|---|---|
| | | Confidence Interval | | | | Confidence Interval | | |
| | | Lower | Upper | | | Lower | Upper | |
| | OR | 95% | 95% | P Value | OR | 95% | 95% | P Value |
| Age (increase per year) | 1.04 | 1.02 | 1.06 | <0.0001 | 1.07 | 1.04 | 1.11 | <0.0001 |
| Sex (F) | 1.44 | 0.91 | 2.30 | 0.120 | 1.02 | 0.54 | 1.91 | 0.953 |
| Glucose (increase | 2.25 | 1.23 | 3.99 | 0.010 | 1.83 | 0.90 | 4.07 | 0.094 |
| per log unit) | | | | | | | | |
| CE | 1.93 | 1.15 | 3.31 | 0.013 | 0.67 | 0.34 | 1.32 | 0.249 |
| Afib | 1.23 | 0.69 | 2.19 | 0.475 | 1.21 | 0.59 | 2.44 | 0.600 |
| CHF | 1.41 | 0.71 | 2.79 | 0.322 | 0.79 | 0.33 | 1.76 | 0.566 |
| IV tPA | 0.57 | 0.35 | 0.93 | 0.024 | 0.82 | 0.43 | 1.55 | 0.537 |
| NIHSS (increase | 5.88 | 4.31 | 8.26 | <0.0001 | 7.27 | 4.54 | 12.46 | <0.0001 |
| per log unit) | | | | | | | | |
| Sample storage time | 1.03 | 1.00 | 1.07 | 0.020 | 0.96 | 0.74 | 1.04 | 0.705 |
| sST2 (increase | 2.90 | 1.60 | 5.32 | 0.002 | 3.69 | 1.62 | 8.92 | 0.001 |
| per log unit) | | | | | | | | |

TABLE 7
sST2 predicts mortality independent from acute
and chronic cardiovascular risk factors.

| | | Confidence Interval | | |
|---|---|---|---|---|
| | | Lower | Upper | |
| | OR | 95% | 95% | P Value |
| Model 1 | 3.83 | 2.13 | 7.21 | <0.0001 |
| Model 2 | 2.98 | 1.41 | 6.56 | 0.002 |
| Model 3 | 4.11 | 1.47 | 12.65 | 0.020 |
| Model 4 | 3.83 | 1.24 | 13.24 | 0.033 |
| Model 5 | 3.83 | 1.27 | 13.68 | 0.033 |
| Model 6 | 3.51 | 1.10 | 12.47 | 0.049 |

Model 1: Unadjusted
Model 2: Adjusted for age, sex, NIHSS, glucose, IV tPA,
Model 3: Adjusted for age, sex, NIHSS, glucose, IV tPA, troponin
Model 4: Adjusted for age, sex, NIHSS, glucose, IV tPA, EF
Model 5: Adjusted for age, sex, NIHSS, glucose, IV tPA, troponin, EF
Model 6: Adjusted for age, sex, NIHSS, glucose, IV tPA, troponin, EF,
Afib, CHF, CE
OR, odds ratio; NIHSS, National Institutes of Health Stroke Scale; IV tPA, intravenous tissue plasminogen activator; EF, ejection fraction, Afib indicates Atrial Fibrillation; CHF, Congestive Heart Failure; CE, cardioembolic stroke subtype

TABLE 8
sST2 predicts hemorrhagic transformation after ischemic stroke - detailed.
Multivariate Analysis

| | | Confidence Interval | | |
|---|---|---|---|---|
| | | Lower | Upper | |
| | OR | 95% | 95% | P Value |
| Model 1 | 5.40 | 1.40 | 35.61 | 0.039 |
| Model 2 | 5.43 | 1.40 | 35.94 | 0.039 |
| Model 3 | 4.74 | 1.21 | 31.54 | 0.024 |
| Model 4 | 5.24 | 1.33 | 34.97 | 0.050 |
| Model 5 | 4.91 | 1.26 | 32.57 | 0.020 |
| Model 6 | 5.20 | 1.33 | 34.50 | 0.050 |
| Model 7 | 5.15 | 1.32 | 34.13 | 0.050 |
| Model 8 | 5.74 | 1.47 | 38.03 | 0.029 |

| | |
|---|---|
| Model 1 | Adjusted for NIHSS, BL DWI, Anticoagulant use |
| Model 2 | Adjusted for NIHSS, BL DWI, Anticoagulant use + age |
| Model 3 | Adjusted for NIHSS, BL DWI, Anticoagulant use + sex |
| Model 4 | Adjusted for NIHSS, BL DWI, Anticoagulant use + glucose |
| Model 5 | Adjusted for NIHSS, BL DWI, Anticoagulant use + IV tPA |
| Model 6 | Adjusted for NIHSS, BL DWI, Anticoagulant use + Smoking |
| | history |
| Model 7 | Adjusted for NIHSS, BL DWI, Anticoagulant use + |
| | Antiplatelet use |
| Model 8 | Adjusted for NIHSS, BL DWI, Anticoagulant use + MMP-9 |

NIHSS indicates National Institutes of Health Stroke Scale; sST2, soluble ST2; IV tPA, intravenous tissue plasminogen activator; MMP-9, matrix metalloproteinase-9; BL DWI, baseline DWI volume.

TABLE 9
48h sST2 predicts poor outcome and mortality after ischemic stroke.

| Univariate Analysis | | | | Multivariate Analysis | | | |
|---|---|---|---|---|---|---|---|
| | Confidence Interval | | | | Confidence Interval | | |
| | Lower | Upper | | | Lower | Upper | |
| OR | 95% | 95% | P Value | OR | 95% | 95% | P Value |

Univariate and multivariate predictors of poor outcome after stroke (mRS 3-6).

| | | | | | | | | |
|---|---|---|---|---|---|---|---|---|
| Age (increase per year) | 1.06 | 1.04 | 1.09 | <0.0001 | 1.05 | 1.01 | 1.10 | 0.018 |
| Sex (F) | 2.02 | 1.27 | 3.22 | 0.003 | 2.55 | 0.91 | 7.48 | 0.077 |
| Glucose (increase | 2.45 | 1.17 | 5.31 | 0.018 | 1.12 | 0.20 | 5.43 | 0.889 |
| per log unit) | | | | | | | | |
| CE | 1.22 | 0.77 | 1.93 | 0.389 | 0.56 | 0.19 | 1.61 | 0.286 |
| Afib | 2.34 | 1.43 | 3.87 | 0.001 | 0.64 | 0.18 | 2.14 | 0.477 |
| CHF | 2.00 | 0.97 | 4.15 | 0.059 | 1.01 | 0.27 | 3.76 | 0.984 |
| IV tPA | 2.40 | 1.51 | 3.85 | 0.001 | 0.75 | 0.27 | 1.96 | 0.557 |
| NIHSS (increase | 7.78 | 5.16 | 12.39 | <0.0001 | 11.53 | 5.62 | 27.20 | <0.0001 |
| per log unit) | | | | | | | | |
| sST2 (increase | 5.84 | 2.98 | 12.52 | <0.0001 | 4.08 | 1.21 | 16.33 | 0.022 |
| per log unit) | | | | | | | | |

Univariate and multivariate predictors of death after stroke.

| | | | | | | | | |
|---|---|---|---|---|---|---|---|---|
| Age (increase per year) | 1.08 | 1.05 | 1.12 | <0.0001 | 1.06 | 1.00 | 1.45 | 0.048 |
| Sex (F) | 1.59 | 0.86 | 2.97 | 0.140 | 1.80 | 0.47 | 7.02 | 0.386 |
| Glucose (increase | 3.51 | 1.44 | 8.45 | 0.006 | 1.11 | 0.14 | 7.92 | 0.919 |
| per log unit) | | | | | | | | |
| CE | 1.91 | 1.03 | 3.66 | 0.041 | 2.21 | 0.53 | 10.26 | 0.279 |
| Afib | 2.91 | 1.55 | 5.49 | 0.001 | 0.52 | 0.10 | 2.29 | 0.396 |
| CHF | 2.58 | 1.10 | 5.69 | 0.030 | 0.63 | 0.11 | 3.00 | 0.568 |
| IV tPA | 1.72 | 0.92 | 3.25 | 0.090 | 0.25 | 0.05 | 1.03 | 0.055 |
| NIHSS (increase | 11.54 | 5.95 | 25.74 | <0.0001 | 11.45 | 3.81 | 48.68 | <0.0001 |
| per log unit) | | | | | | | | |
| sST2 (increase | 9.18 | 3.99 | 24.53 | <0.0001 | 4.01 | 1.30 | 14.95 | 0.015 |
| log unit) | | | | | | | | |

TABLE 10
Cohort characteristics.

| | MGH aSAH cohort | | MGH pSAH cohort | |
|---|---|---|---|---|
| | N = 169 | | N = 17 | |
| age (yrs.) | 57 ± 12 | | 58 ± 8 | |
| sex (F) | 108 | (64%) | 7 | (41%) |
| HH grade | 2 | [1, 4] | 1 | [1, 2] |
| modified Fisher score | 3 | [3, 4] | 3 | [2, 3] |
| Clipping | 73 | (43%) | 0 | (0%) |
| mRS, 90 day | 2 | [1, 4] | 1 | [0, 1] |
| DCI | 61 | (47%) | 0 | (0%) |

| | | |
|---|---|---|
| age (yrs.) | 57 ± 12 | 58 ± 8 |

TABLE 11
sST2 predicts poor outcome (mRS 3-6)
independent of clinical risk factors.

| | | Confidence Interval | | |
|---|---|---|---|---|
| | | Lower | Upper | |
| | OR | 95% | 95% | P value |
| sST2* | 3.02 | 1.58 | 5.76 | 0.001 |
| +age, sex, HH | 2.50 | 1.22 | 5.11 | 0.012 |
| +age, sex, HH, mF | 2.43 | 1.17 | 5.06 | 0.017 |
| +age, sex, HH, mF, clip | 2.58 | 1.20 | 5.56 | 0.015 |
| +age, sex, HH, mF, clip, CHF | 2.56 | 1.19 | 5.52 | 0.017 |
| +age, sex, HH, mF, clip, CHF, | 2.54 | 1.17 | 5.49 | 0.018 |
| Afib | | | | |

*Measured at day 3.5

TABLE 12
sST2 predicts mortality independent of clinical risk factors.

| | | Confidence Interval | | |
|---|---|---|---|---|
| | | Lower | Upper | |
| | OR | 95% | 95% | P value |
| sST2* | 4.62 | 1.92 | 11.10 | 0.001 |
| +age, sex, HH | 4.43 | 1.56 | 12.56 | 0.005 |
| +age, sex, HH, mF | 4.38 | 1.49 | 12.86 | 0.007 |
| +age, sex, HH, mF, clip | 4.13 | 1.38 | 12.40 | 0.011 |
| +age, sex, HH, mF, clip, CHF | 3.90 | 1.30 | 11.73 | 0.015 |
| +age, sex, HH, mF, clip, CHF, | 3.79 | 1.24 | 11.64 | 0.020 |
| Afib | | | | |

*Measured at day 3.5

TABLE 13
Copenhagen replication cohort

| | Copenhagen aSAH cohort N = 50 | |
|---|---|---|
| age (yrs) | 61 ± 11 | |
| sex (F) | 45 | (90%) |
| WFNS grade | 3 | [1, 4] |
| modified Fisher score | 3 | [3, 3] |
| Clipping | 14 | (28%) |
| mRS, 90 day | 1 | [0, 3] |
| DCI | 25 | (50%) |

1)-62) (canceled)

63) A method to identify a subject at risk of a poor functional prognosis after a brain injury, the method comprising;
a. obtaining a biological sample from a subject who has had a brain injury; and
b. measuring a level of sST2 in the biological sample;
wherein the subject is considered at risk of a hemorrhagic transformation when the level of sST2 in the biological sample is increased as compared to the reference level.

64) The method of claim 63, wherein the level of sST2 is increased at least 2-fold, at least 3-fold, at least 4-fold, at least 5-fold, at least 6-fold, at least 7-fold, at least 8-fold, at least 9-fold, at least 10-fold, or more as compared to a reference level.

65) A method to identify a subject at risk of a poor functional prognosis after an ischemic stroke, the method comprising;
a. obtaining a biological sample from a subject who has had an ischemic stroke; and
b. measuring a level of sST2 in the biological sample;
wherein the subject is considered at risk of a poor functional prognosis when the level of sST2 is equal to or greater than 85 ng/mL.

66) A method to identify a subject at risk of a poor functional prognosis after a subarachnoid hemorrhage, the method comprising;
a. obtaining a biological sample from a subject who has had a subarachnoid hemorrhage; and
b. measuring a level of sST2 in the biological sample;
wherein the subject is considered at risk of poor functional prognosis when the level of sST2 is equal to or greater than 50 ng/mL.

67) A method to identify a subject at risk of a hemorrhagic transformation following a brain injury, the method comprising;
a. obtaining a biological sample from a patient who has had a brain injury; and
b. measuring a level of sST2 in the biological sample;
wherein the subject is considered at risk of a hemorrhagic transformation when the level of sST2 in the biological sample is increased as compared to the reference level.

68) The method of claim 65, wherein the level of sST2 is increased at least 2-fold, at least 3-fold, at least 4-fold, at least 5-fold, at least 6-fold, at least 7-fold, at least 8-fold, at least 9-fold, at least 10-fold, or more as compared to a reference level.

69) The method of claim 63, further comprising administering to a subject at risk of a poor function prognosis a composition comprising an agent that inhibitors sST2, or a composition comprising an agent that upmodulates IL-35.

70) The method of claim 63, further comprising administering to a subject at risk of a poor function prognosis a prophylactic treatment.

71)-81) (canceled)


SEQUENCE LISTING (3 sequences)

SEQ ID NO: 1 - DNA, 576 bp, Homo sapiens
atgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttctga

SEQ ID NO: 2 - DNA, 687 bp, Homo sapiens
atgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttag

SEQ ID NO: 3 - protein, 211 aa, Artificial Sequence
  Description of Artificial Sequence Synthetic polypeptide
MYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF